(19) United States
(12) Patent Application Publication
(10) Pub. No.: US 2014/0154264 A1
(43) Pub. Date: Jun. 5, 2014

(54) COMPOSITIONS AND METHODS FOR TREATING CANCER AND DISEASES AND CONDITIONS RESPONSIVE TO CELL GROWTH INHIBITION

(71) Applicant: THE REGENTS OF THE UNIVERSITY OF CALIFORNIA, Okland, CA (US)

(72) Inventors: David Cheresh, Encinitas, CA (US); Aleksandra Franovic, San Diego, CA (US); Laetitia Seguin, La Jolla, CA (US)

(73) Assignee: THE REGENTS OF THE UNIVERSITY OF CALIFORNIA, OAKLAND, CA (US)

(21) Appl. No.: 14/119,078

(22) PCT Filed: Jun. 1, 2012
(86) PCT No.: PCT/US2012/040390

§ 371 Date: Feb. 12, 2014

Related U.S. Application Data
(60) Provisional application No. 61/492,673, filed on Jun. 2, 2011.
(60) Provisional application No. 61/552,881, filed on Oct. 28, 2011.
(60) Provisional application No. 61/620,725, filed on Apr. 5, 2012.

(51) Int. Cl. A61K 31/517; G01N 33/574

(52) U.S. Cl. CPC A61K 31/517; G01N 33/574
USPC 424/158.1; 514/266.4; 435/7.23; 435/184

(57) ABSTRACT

In alternative embodiments, the invention provides compositions and methods for identifying individuals that would be responsive to a treatment comprising (including) blocking activation of integrin polypeptide alphaᵥ-beta₃ (or αᵥ-β₃), or blocking the interaction of a ligand with integrin polypeptide alphaᵥ-beta₃ (or αᵥ-β₃). The invention provides compositions and methods for determining the effectiveness of such a treatment and can contribute to a prognosis for the patient.

### GOVERNMENT RIGHTS

This invention was made with government support under grant numbers CA045726, CA050286, CA095262, HL057900, and HL103956, awarded by the National Institutes of Health (NIH). The government has certain rights in the invention.

### TECHNICAL FIELD

This invention generally relates to cell biology, diagnostics, personalized medicine and oncology. In alternative embodiments, the invention provides compositions and methods for identifying individuals that would be responsive to a treatment comprising (including) blocking activation of integrin polypeptide alphaᵥ-beta₃ (or αᵥ-β₃), or blocking the interaction of a ligand with integrin polypeptide alphaᵥ-beta₃ (or αᵥ-β₃). The invention provides compositions and methods for determining the effectiveness of such a treatment and can contribute to a prognosis for the patient.

In alternative embodiments, the invention provides methods for regulating or modulating RAF kinases. In alternative embodiments, the invention provides compositions and methods for: arresting a proliferating tumor cell at prometaphase by reducing or inhibiting the activity of a human P21 protein (Cdc42/Rac)-Activated Kinase (PAK or c-PAK); reducing or inhibiting serine 338 (Ser 338) phosphorylation of a c-RAF; reducing or inhibiting a c-RAF-dependent dysfunctional cell, cancer cell or tumor growth; promoting a tumor regression in vivo in a c-RAF-dependent human tumor or cancer cell; inducing double-stranded DNA breakage in a cell; or, sensitizing a tumor cell to a radiation (radiosensitizing a cell) or a chemotherapy; comprising providing a composition comprising or consisting of an inhibitor, e.g., a direct inhibitor, of a PAK (or c-PAK) protein activity.

This invention generally relates to cell and molecular biology, diagnostics and oncology. In alternative embodiments, the invention provides compositions and methods for overcoming or diminishing or preventing Growth Factor Inhibitor resistance in a cell, or, a method for increasing the growth-inhibiting effectiveness of a Growth Factor inhibitor on a cell, or, a method for re-sensitizing a cell to a Growth Factor Inhibitor. In alternative embodiments, the cell is a tumor cell, a cancer cell, a cancer stem cell or a dysfunctional cell. In alternative embodiments, the invention provides compositions and methods for determining: whether an individual or a patient would benefit from or respond to administration of a Growth Factor Inhibitor, or, which individuals or patients would benefit from a combinatorial approach comprising administration of a combination of: at least one growth factor and at least one compound, composition or formulation used to practice a method of the invention, such as an NfKb inhibitor.

### BACKGROUND

Growth factor inhibitors have been used to treat many cancers including pancreatic, breast, lung and colorectal cancers. However, resistance to growth factor inhibitors has emerged as a significant clinical problem.

RAF (Raf) is a serine/threonine protein kinase that phosphorylates the OH group of serine or threonine. c-Raf is a MAP kinase (MAP3K) which functions downstream of the Ras subfamily of membrane associated GTPases to which it binds directly. Once activated Raf-1 can phosphorylate to activate the dual specificity protein kinases MEK1 and MEK2 which in turn phosphorylate to activate the serine/threonine specific protein kinases ERK1 and ERK2. RAF kinases play a role in tumorigenesis, and are associated with tumor metastasis, radiation and chemo-resistance, and angiogenesis.

p21 activated kinases (PAKs) are a family of serine/threonine p21-activated kinases, include PAK1, PAK2, PAK3 and PAK4, implicated in a wide range of biological activities. PAKs are protein effectors that can link RhoGTPases to cytoskeleton reorganization and nuclear signaling. PAKs are members of a family of enzyme that can be targets for GTP binding proteins such as CDC42 and RAC. PAK members include: PAK1, known to regulate cell motility and morphology; PAK2, which possibly plays a role in apoptosis; PAK3, which possibly play a role in dendritic development and cytoskeletal reorganization in dendritic spines associated with synaptic plasticity.

RAF (Rat) is a serine/threonine protein kinase that phosphorylates the OH group of serine or threonine. c-Raf is a MAP kinase (MAP3K) which functions downstream of the Ras subfamily of membrane associated GTPases to which it binds directly (RAF proto-oncogene serine/threonine-protein kinase is also known as proto-oncogene c-RAF or simply c-Rat). Once activated Raf-1 can phosphorylate to activate the dual specificity protein kinases MEK1 and MEK2 which in turn phosphorylate to activate the serine/threonine specific protein kinases ERK1 and ERK2. RAF kinases play a role in tumorigenesis, and are associated with tumor metastasis, radiation and chemo-resistance, and angiogenesis. RAF kinases regulate cell proliferation and survival and can be dysregulated in tumors. A role for RAF in cell proliferation has been linked to its ability to activate MEK and ERK.

### SUMMARY

In alternative embodiments, the invention provides compositions and methods for identifying (or determining whether) an individual would be (or has a substantial likelihood of being) responsive to a treatment comprising (e.g., a treatment involving or including) blocking activation of an alphaᵥ-beta₃ (or αᵥ-β₃) integrin polypeptide, or blocking the interaction of a ligand with an alphaᵥ-beta₃ (or αᵥ-β₃) integrin polypeptide, or blocking the phosphorylation of a C-RAF polypeptide, comprising:

identifying (determining) the phosphorylation state of a C-RAF serine residue 338 (ser-338) on a C-RAF polypeptide, wherein identifying (determining) that a C-RAF serine residue 338 (ser-338) is phosphorylated, or identifying (determining) the extent to which cellular C-RAFs are ser-338 phosphorylated, and a finding (identification or determination) that ser-338 residues are phosphorylated identifies or determines that the individual will be or has a substantial likelihood of being responsive to a treatment comprising blocking activation of an alphaᵥ-beta₃ (or αᵥ-β₃) integrin polypeptide, or blocking the interaction of a ligand with an alphaᵥ-beta₃ (or αᵥ-β₃) integrin polypeptide, or blocking the phosphorylation of a C-RAF polypeptide.

In alternative embodiments, the individual is a human, and/or the treatment is for a cancer, a carcinoma, a pancreatic carcinoma, a lung carcinoma, a laryngeal carcinoma, a melanoma, a brain cancer or tumor or a glioblastoma, and/or the treatment is for inhibiting or impeding blood vessel growth (anti-angiogenic) or anti-metastatic.

In alternative embodiments, the treatment comprises administration of: a cyclic RGD peptide, or cilengitide (Merck KGaA, Darmstadt, Germany); or any small molecule, peptide, polypeptide or antibody that blocks activation of an alphaᵥ-beta₃ (or αᵥ-β₃) integrin polypeptide, or blocks the interaction of a ligand with an alphaᵥ-beta₃ (or αᵥ-β₃) integrin polypeptide, or blocks the phosphorylation of a C-RAF polypeptide, or blocks the phosphorylation of a C-RAF serine residue 338 (ser-338).

In alternative embodiments, a finding (identification or determination) that ser-338 residues are substantially phosphorylated, or phosphorylated to a greater degree than ound in a wild type or normal cell, identifies or determines that the individual will be or has a substantial likelihood of being responsive to a treatment comprising blocking activation of an alphaᵥ-beta₃ (or αᵥ-β₃) integrin polypeptide, or blocking the interaction of a ligand with an alphaᵥ-beta₃ (or αᵥ-β₃) integrin polypeptide, or blocking the phosphorylation of a C-RAF polypeptide.

In alternative embodiments, these determinations/findings are used to design a treatment regimen, e.g., whether additional or ancillary treatments will be necessary, e.g., radiation and/or chemotherapies, and/or what dosages of drugs should be administered.

The invention provides compositions and methods for determining the responsiveness of an individual to a treatment (or the effectiveness of the treatment) comprising (e.g., a treatment involving or including) blocking activation of an alphaᵥ-beta₃ (or αᵥ-β₃) integrin polypeptide, or blocking the interaction of a ligand with an alphaᵥ-beta₃ (or αᵥ-β₃) integrin polypeptide, or blocking the phosphorylation of a C-RAF polypeptide, comprising:

identifying (determining) the phosphorylation state of a C-RAF serine residue 338 (ser-338) on a C-RAF polypeptide, or identifying (determining) that a C-RAF serine residue 338 (ser-338) is phosphorylated, or identifying (determining) the extent to which cellular C-RAFs are ser-338 phosphorylated, determines the responsiveness of an individual to the treatment.

In alternative embodiments, the method comprises identifying (determining) the phosphorylation state of a C-RAF serine residue 338 (ser-338) on a C-RAF polypeptide, or identifying (determining) that a C-RAF serine residue 338 (ser-338) is phosphorylated, or identifying (determining) the extent to which cellular C-RAFs are ser-338 phosphorylated, at two different time points, and if the amount of phosphorylation of ser-338 decreases in the second time point relative to the first time point, the individual is determined to be responsive to the treatment.

In alternative embodiments, the method is prognostic in that individuals (e.g., patients) having decreased levels (amounts) of phosphorylated ser-338 are determined or predicted to survive longer. In alternative embodiments, these determinations/findings are used to design a treatment regimen, e.g., whether additional or ancillary treatments will be necessary, e.g., radiation and/or chemotherapies, and/or what dosages of drugs should be administered.

In alternative embodiments, the invention provides compositions and methods for:
arresting a proliferating tumor cell at prometaphase by reducing or inhibiting the activity of a human P21 protein (Cdc42/Rac)-Activated Kinase (PAK or c-PAK);
reducing or inhibiting serine 338 (Ser 338) phosphorylation of a c-RAF;
reducing or inhibiting a c-RAF-dependent dysfunctional cell, cancer cell or tumor growth;
promoting a tumor regression in vivo in a c-RAF-dependent human tumor or cancer cell;
inducing double-stranded DNA breakage in a cell; or,
sensitizing a tumor cell to a radiation (radiosensitizing a cell) or a chemotherapy; comprising

(1) (a) providing a composition comprising or consisting of:
(i) an inhibitor of a PAK (or c-PAK) protein activity, or
(ii) the PAK-inhibiting composition of (i), wherein the PAK inhibitor comprises a small molecule, an antibody, a dominant negative PAK inhibitor, a siRNA, an miRNA, or an antisense oligonucleotide; and

(b) administering a sufficient amount of the composition to a cell or a subject to reduce or inhibit the activity of the PAK kinase, or human PAK kinase,

wherein optionally administering the PAK inhibitor comprises arresting a proliferating tumor cell at prometaphase,

wherein optionally administering the PAK inhibitor comprises reducing or inhibiting a serine 338 (Ser 338) phosphorylation of a c-RAF,

wherein optionally administering the PAK inhibitor reduces or inhibits a c-RAF-dependent dysfunctional cell, cancer cell or tumor growth,

wherein optionally administering the PAK inhibitor promotes a tumor regression in vivo in a c-RAF-dependent human tumor or cancer cell,

wherein optionally administering the PAK inhibitor induces double-stranded DNA breakage in a cell, or sensitizes a tumor cell to a radiation or a chemotherapy; or

(2) the method of (1), wherein the composition comprises a pharmaceutical composition formulated for administration in vivo;

(3) the method of (1) or (2), wherein the composition is formulated for administration intravenously (IV), parenterally, nasally, topically, orally, or by liposome or vessel-targeted nanoparticle delivery;

(4) the method of any of (1) to (3), wherein the composition comprises a pharmaceutical composition administered in vivo;

(5) the method of any of (1) to (3), wherein the administration comprises contacting a cell or tumor in vitro or ex vivo;

(6) the method of any of (1) to (5), wherein the dominant-negative peptide PAK inhibitor comprises a peptidomimetic;

(7) the method of any of (1) to (5), wherein the PAK inhibitor comprises or consists of a peptide having a sequence HTIHVGFDAV TGEFTGMPEQ WARLLQTSNI TKSEQKKNPQ AVLDVLEFYN SKKTSNSQKY MSFTDKS (SEQ ID NO:1), or as described in U.S. Pat. No. 7,364,887;

(8) the method of any of (1) to (5), wherein the antibody PAK inhibitor comprises or is a monoclonal antibody, a humanized antibody or a human antibody, or an antigen-binding (PAK-binding) fragment thereof; or

(9) the method of any of (1) to (8), wherein the method reduces, treats or ameliorates the level of disease in a retinal age-related macular degeneration, a diabetic retinopathy, a cancer or carcinoma, a glioblastoma, a neuroma, a neuroblastoma, a colon carcinoma, a hemangioma, an infection and/or a condition with at least one inflammatory component, and/or any infectious or inflammatory disease, such as a rheumatoid arthritis, a psoriasis, a fibrosis, leprosy, multiple sclerosis, inflammatory bowel disease, or ulcerative colitis or Crohn's disease.

In alternative embodiments, the invention provides compositions and methods for reducing, treating or ameliorating a condition or disease responsive to slowing, decreasing the rate of, arresting or inhibiting cell growth, comprising:

(1) (a) providing a composition comprising or consisting of:
(i) an inhibitor of a PAK (or c-PAK) protein activity, or
(ii) the PAK-inhibiting composition of (i), wherein the PAK inhibitor comprises a small molecule, an antibody, a dominant negative PAK inhibitor, a siRNA, an miRNA, or an antisense oligonucleotide; and

(b) administering a sufficient amount of the composition to a cell or a subject to reduce or inhibit the activity of the PAK kinase, or human PAK kinase,

thereby reducing, treating or ameliorating the condition or disease responsive to slowing, decreasing the rate of, arresting or inhibiting cell growth;

(2) the method of (1), wherein the composition comprises a pharmaceutical composition formulated for administration in vivo;

(3) the method of (1) or (2), wherein the composition is formulated for administration intravenously (IV), parenterally, nasally, topically, orally, or by liposome or vessel-targeted nanoparticle delivery;

(4) the method of any of (1) to (3), wherein the composition comprises a pharmaceutical composition administered in vivo;

(5) the method of any of (1) to (3), wherein the administration comprises contacting a cell or tumor in vitro or ex vivo;

(6) the method of any of (1) to (5), wherein the dominant-negative peptide PAK inhibitor comprises a peptidomimetic;

(7) the method of any of (1) to (5), wherein the PAK inhibitor comprises or consists of a peptide having a sequence HTIHVGFDAV TGEFTGMPEQ WARLLQTSNI TKSEQKKNPQ AVLDVLEFYN SKKTSNSQKY MSFTDKS (SEQ ID NO:1), or as described in U.S. Pat. No. 7,364,887;

(8) the method of any of (1) to (5), wherein the antibody PAK inhibitor comprises or is a monoclonal antibody, a humanized antibody or a human antibody, or an antigen-binding (PAK-binding) fragment thereof; or

(9) the method of any of (1) to (8), wherein the method reduces, treats or ameliorates the level of disease in a retinal age-related macular degeneration, a diabetic retinopathy, a cancer or carcinoma, a glioblastoma, a neuroma, a neuroblastoma, a colon carcinoma, a hemangioma, an infection and/or a condition with at least one inflammatory component, and/or any infectious or inflammatory disease, such as a rheumatoid arthritis, a psoriasis, a fibrosis, leprosy, multiple sclerosis, inflammatory bowel disease, or ulcerative colitis or Crohn's disease.

In alternative embodiments, the invention provides kits comprising a composition comprising or consisting of: (i) an inhibitor of a PAK (or c-PAK) protein activity, wherein optionally the PAK inhibitor comprises a small molecule, an antibody, a dominant negative PAK inhibitor, a siRNA, an miRNA, or an antisense oligonucleotide; and (ii) instructions for practicing a method of the invention.

In alternative embodiments, the invention provides compositions and methods for determining whether an individual or a patient would benefit from administration of an inhibitor of a PAK (or c-PAK) protein activity, comprising:

(a) detecting a serine-338 phosphorylated c-RAF, or detecting a serine-338 phosphorylated c-RAF localized to the mitotic spindle, wherein optionally the detection is by analysis or visualization of a biopsy or other tissue sample or a pathology slide taken from the patient or individual,

wherein detection of a serine-338 phosphorylated c-RAF, or detection of a serine-338 phosphorylated c-RAF localized to the mitotic spindle, indicates: that the individual or patient will be responsive to the inhibitor of a PAK (or c-PAK) protein activity.

In alternative embodiments, the invention provides compositions and methods for determining whether an individual, subject or a patient would benefit from administration of an inhibitor of a PAK (or c-PAK) protein activity, comprising:

(a) providing a composition comprising or consisting of:
(i) an inhibitor of a PAK (or c-PAK) protein activity, or
(ii) the PAK-inhibiting composition of (i), wherein the PAK inhibitor comprises a small molecule, an antibody, a dominant negative PAK inhibitor, a siRNA, an miRNA, or an antisense oligonucleotide,

wherein optionally the levels of serine-338 phosphorylated c-RAF, or detection of a serine-338 phosphorylated c-RAF localized to the mitotic spindle, is measured or determined before administering the composition to a cell, a tissue, a tumor or an individual or subject; and

(b) administering the composition to a cell, a tissue, a tumor or an individual,

(c) detecting an increase or a decrease in serine-338 phosphorylated c-RAF, or detecting an increase or a decrease in serine-338 phosphorylated c-RAF localized to the mitotic spindle,

wherein optionally the detection is by analysis or visualization of a biopsy or other tissue sample or a pathology slide taken from the patient or individual,

wherein detection of a decrease in the serine-338 phosphorylated c-RAF, or detection of a decrease in the serine-338 phosphorylated c-RAF localized to the mitotic spindle, indicates: that the individual or patient will be responsive to the inhibitor of a PAK (or c-PAK) protein activity.

In alternative embodiments, the invention provides compositions and methods for inducing double-stranded DNA breakage in a cell, or for sensitizing a tumor cell, a tumor, a metastasis or a subject to a radiation or a chemotherapy comprising:

(a) providing a composition comprising or consisting of:
(i) an inhibitor of a PAK (or c-PAK) protein activity, or
(ii) the PAK-inhibiting composition of (i), wherein the PAK inhibitor comprises a small molecule, an antibody, a dominant negative PAK inhibitor, a siRNA, an miRNA, or an antisense oligonucleotide; and

(b) administering a sufficient amount of the composition to induce double-stranded DNA breakage in the cell, or to sensitize the tumor cell, tumor, metastasis or subject to the radiation or the chemotherapy.

In alternative embodiments, the invention provides compositions and methods for arresting a proliferating tumor cell at prometaphase comprising:

(a) providing a composition comprising or consisting of:
(i) an inhibitor of a PAK (or c-PAK) protein activity, or
(ii) the PAK-inhibiting composition of (i), wherein the PAK inhibitor comprises a small molecule, an antibody, a dominant negative PAK inhibitor, a siRNA, an miRNA, or an antisense oligonucleotide; and

(b) administering a sufficient amount of the composition to the proliferating tumor cell to arrest the proliferating tumor cell at prometaphase.

In alternative embodiments, the invention provides uses of a compound in the preparation of a medicament for
arresting a proliferating tumor cell at prometaphase by reducing or inhibiting the activity of a human P21 protein (Cdc42/Rac)-Activated Kinase (PAK or c-PAK);
reducing or inhibiting serine 338 (Ser 338) phosphorylation of a c-RAF;
reducing or inhibiting a c-RAF-dependent dysfunctional cell, cancer cell or tumor growth;
promoting a tumor regression in vivo in a c-RAF-dependent human tumor or cancer cell; or,
sensitizing a tumor cell to a radiation or a chemotherapy, comprising: use of a composition comprising or consisting of:
(i) an inhibitor of a PAK (or c-PAK) protein activity, or
(ii) the PAK-inhibiting composition of (i), wherein the PAK inhibitor comprises a small molecule, an antibody, a dominant negative PAK inhibitor, a siRNA, an miRNA, or an antisense oligonucleotide.

In alternative embodiments, the invention provides uses wherein a sufficient amount of the composition is administered to the cell to arrest the proliferating tumor cell at prometaphase, or to regulate or modulate cell growth or mitosis, or induce double-stranded DNA breakage in the cell, or to sensitize a tumor cell, tumor, metastasis or subject to a radiation or a chemotherapy,

the use of the compound, or the medicament, reduces, treats or ameliorates the level of disease in a retinal age-related macular degeneration, a diabetic retinopathy, a cancer or carcinoma, a glioblastoma, a neuroma, a neuroblastoma, a colon carcinoma, a hemangioma, an infection and/or a condition with at least one inflammatory component, and/or any infectious or inflammatory disease, such as a rheumatoid arthritis, a psoriasis, a fibrosis, leprosy, multiple sclerosis, inflammatory bowel disease, or ulcerative colitis or Crohn's disease.

In alternative embodiments, the invention provides methods for overcoming or diminishing or preventing Growth Factor Inhibitor (GFI) resistance in a cell, or, a method for increasing the growth-inhibiting effectiveness of a Growth Factor inhibitor on a cell, or, a method for re-sensitizing a cell to a Growth Factor Inhibitor (GFI),

wherein optionally the cell is a tumor cell, a cancer cell, a cancer stem cell, or a dysfunctional cell,

the method comprising:

(1) (a) providing at least one compound, composition or formulation comprising or consisting of:
(i) an inhibitor or depleter of integrin αᵥβ₃ (anb3), or an inhibitor of integrin αᵥβ₃ (anb3) protein activity, or an inhibitor of the formation or activity of an integrin anb3/RalB signaling complex, or an inhibitor of the formation or signaling activity of an integrin αᵥβ₃ (anb3)/RalB/NFkB signaling axis,
wherein the inhibitor of integrin αᵥβ₃ protein activity is an allosteric inhibitor of integrin αᵥβ₃ protein activity;
(ii) an inhibitor or depleter of RalB protein or an inhibitor of RalB protein activation,
wherein the inhibitor of RalB protein activity is an allosteric inhibitor of RalB protein activity;
(iii) an inhibitor or depleter of Src or TBK1 protein or an inhibitor of Src or TBK1 protein activation,
wherein the inhibitor of Src or TBK1 protein activity is an allosteric inhibitor of Src or TBK1 protein activity;
(iv) an inhibitor or depleter of NFKB or IRF3 protein or an inhibitor of RalB protein activation,
wherein the inhibitor of NFKB or IRF3 protein activity is an allosteric inhibitor of NFKB or IRF3 protein activity; or
(v) any combination of (i) to (iv); and

(b) administering a sufficient amount of the at least one compound, composition or formulation to the cell to: overcome or diminish or prevent Growth Factor Inhibitor (GFI) resistance in the cell, or, increase the growth-inhibiting effectiveness of a Growth Factor Inhibitor on the cell, or, re-sensitize the cell to the Growth Factor Inhibitor (GFI).

In alternative embodiments of the methods:

(a) the at least one compound, composition or formulation is a pharmaceutical composition;

(b) the method of (a), wherein the compound, composition or formulation or pharmaceutical composition is administered in vitro, ex vivo or in vivo, or is administered to an individual in need thereof;

(c) the method of (a) or (b), wherein the at least one compound, composition or formulation is a pharmaceutical composition is formulated for administration intravenously (IV), parenterally, nasally, topically, orally, or by liposome or targeted or vessel-targeted nanoparticle delivery;

(d) the method of any of (a) to (c), wherein the compound or composition comprises or is an inhibitor of transcription, translation or protein expression;

(e) the method of any of (a) to (d), wherein the compound or composition is a small molecule, a protein, an antibody, a monoclonal antibody, a nucleic acid, a lipid or a fat, a polysaccharide, an RNA or a DNA;

(f) the method of any of (a) to (e), wherein the compound or composition comprises or is: a VITAXIN™ (Applied Molecular Evolution, San Diego, Calif.) antibody, a humanized version of an LM609 monoclonal antibody, an LM609 monoclonal antibody, or any antibody that functionally blocks an αᵥβ₃ integrin or any member of an αᵥβ₃ integrin-comprising complex or an integrin αᵥβ₃ (anb3)/RalB/NFkB signaling axis;

(g) the method of any of (a) to (e), wherein the compound or composition comprises or is a Src inhibitor or an NFkB inhibitor;

(h) the method of any of (1) to (5), wherein Growth Factor Inhibitor is or comprises an anti-metabolite inhibitor, a gemcitabine, GEMZAR™, a mitotic poison, a paclitaxel, a taxol, ABRAXANE™, an erlotinib, TARCEVA™, a lapatinib, TYKERB™, or an insulin growth factor inhibitor;

(i) the method of any of (1) to (5), wherein the Growth Factor Inhibitor decreases, slows or blocks new blood vessel growth, neovascularization or angiogenesis; or, wherein administering the Growth Factor Inhibitor treats or ameliorates conditions that are responsive to blocking or slowing cell growth, and/or the development of neovascularization or new blood vessels; or

(j) wherein the method reduces, treats or ameliorates the level of disease in a retinal age-related macular degeneration, a diabetic retinopathy, a cancer or carcinoma, a glioblastoma, a neuroma, a neuroblastoma, a colon carcinoma, a hemangioma, an infection and/or a condition with at least one inflammatory component, and/or any infectious or inflammatory disease, such as a rheumatoid arthritis, a psoriasis, a fibrosis, leprosy, multiple sclerosis, inflammatory bowel disease, or ulcerative colitis or Crohn's disease.

In alternative embodiments, the invention provides kits, blister packages, lidded blisters or blister cards or packets, clamshells, trays or shrink wraps, comprising;

(a) (i) at least one compound, composition or formulation used to practice a method of the invention, and (ii); at least one Growth Factor Inhibitor; or

(b) the kit of (a), further comprising instructions for practicing a method of the invention.

In alternative embodiments, the invention provides methods for determining: whether an individual or a patient would benefit from or respond to administration of a Growth Factor Inhibitor, or

which individuals or patients would benefit from a combinatorial approach comprising administration of a combination of: at least one growth factor and at least one compound, composition or formulation used to practice a method of the invention, such as an NfKb inhibitor,

the method comprising:

detecting the levels or amount of integrin αᵥβ₃ (anb3) and/or active RalB complex in or on a cell, a tissue or a tissue sample,

wherein optionally the detection is by analysis or visualization of a biopsy or a tissue, urine, fluid, serum or blood sample, or a pathology slide taken from the patient or individual, or by a fluorescence-activated cell sorting (FACS) or flow cytometry analysis or the sample or biopsy,

wherein optionally the cell or tissue or tissue sample is or is derived from a tumor or a cancer,

wherein optionally the method further comprises taking a biopsy or a tissue, urine, fluid, serum or blood sample from an individual or a patient,

wherein a finding of increased levels or amounts of integrin αᵥβ₃ (anb3) and/or active RalB complexes in or on the cell, tissue or the tissue sample as compared to normal, normalized or wild type cells or tissues, indicates that:

the individual or patient would benefit from a combinatorial approach comprising administration of a combination of: at least one growth factor and at least one compound, composition or formulation used to practice a method of the invention.

In alternative embodiments of methods of the invention, the detecting of the levels or amount of integrin αᵥβ₃ (anb3) and/or active RalB complex in or on the cell, tissue or the tissue sample is done before or during a drug or a pharmaceutical treatment of an individual using at least one compound, composition or formulation used to practice a method of the invention.

In alternative embodiments, the invention provide uses of a combination of compounds in the manufacture of a medicament,

wherein the combination of compounds comprises:

(1) at least one compound comprising or consisting of:
(i) an inhibitor or depleter of integrin αᵥβ₃ (anb3), or an inhibitor of integrin αᵥβ₃ (anb3) protein activity, or an inhibitor of the formation or activity of an integrin anb3/RalB signaling complex, or an inhibitor of the formation or signaling activity of an integrin αᵥβ₃ (anb3)/RalB/NFkB signaling axis,
wherein the inhibitor of integrin αᵥβ₃ protein activity is an allosteric inhibitor of integrin αᵥβ₃ protein activity;
(ii) an inhibitor or depleter of RalB protein or an inhibitor of RalB protein activation,
wherein the inhibitor of RalB protein activity is an allosteric inhibitor of RalB protein activity;
(iii) an inhibitor or depleter of Src or TBK1 protein or an inhibitor of Src or TBK1 protein activation,
wherein the inhibitor of Src or TBK1 protein activity is an allosteric inhibitor of Src or TBK1 protein activity;
(iv) an inhibitor or depleter of NFKB or IRF3 protein or an inhibitor of RalB protein activation,
wherein the inhibitor of NFKB or IRF3 protein activity is an allosteric inhibitor of NFKB or IRF3 protein activity; or
(v) any combination of (i) to (iv); and

(2) at least one Growth Factor Inhibitor.

In alternative embodiments, the invention provides therapeutic combinations of drugs comprising or consisting of a combination of at least two compounds: wherein the at least two compounds comprise or consist of:

(1) at least one compound comprising or consisting of:
(i) an inhibitor or depleter of integrin αᵥβ₃ (anb3), or an inhibitor of integrin αᵥβ₃ (anb3) protein activity, or an inhibitor of the formation or activity of an integrin anb3/RalB signaling complex, or an inhibitor of the formation or signaling activity of an integrin αᵥβ₃. (anb3)/RalB/NFkB signaling axis,
wherein the inhibitor of integrin αᵥ/β₃ protein activity is an allosteric inhibitor of integrin αᵥβ₃ protein activity;
(ii) an inhibitor or depleter of RalB protein or an inhibitor of RalB protein activation,
wherein the inhibitor of RalB protein activity is an allosteric inhibitor of RalB protein activity;
(iii) an inhibitor or depleter of Src or TBK1 protein or an inhibitor of Src or TBK1 protein activation,
wherein the inhibitor of Src or TBK1 protein activity is an allosteric inhibitor of Src or TBK1 protein activity;
(iv) an inhibitor or depleter of NFKB or IRF3 protein or an inhibitor of RalB protein activation,
wherein the inhibitor of NFKB or IRF3 protein activity is an allosteric inhibitor of NFKB or IRF3 protein activity; or
(v) any combination of (i) to (iv); and

(2) at least one Growth Factor Inhibitor.

In alternative embodiments, the invention provides combinations, or therapeutic combinations, for overcoming or diminishing or preventing Growth Factor Inhibitor (GFI) resistance in a cell, or, a method for increasing the growth-inhibiting effectiveness of a Growth Factor inhibitor on a cell, or, a method for re-sensitizing a cell to a Growth Factor Inhibitor (GFI), wherein the combination comprises or consists of:

(1) at least one compound comprising or consisting of:
(i) an inhibitor or depleter of integrin αᵥβ₃ (anb3), or an inhibitor of integrin αᵥβ₃ (anb3) protein activity, or an inhibitor of the formation or activity of an integrin anb3/RalB signaling complex, or an inhibitor of the formation or signaling activity of an integrin αᵥβ₃ (an 63)/RalB/NFkB signaling axis,
wherein the inhibitor of integrin αᵥβ₃ protein activity is an allosteric inhibitor of integrin αᵥβ₃ protein activity;
(ii) an inhibitor or depleter of RalB protein or an inhibitor of RalB protein activation,
wherein the inhibitor of RalB protein activity is an allosteric inhibitor of RalB protein activity;
(iii) an inhibitor or depleter of Src or TBK1 protein or an inhibitor of Src or TBK1 protein activation,
wherein the inhibitor of Src or TBK1 protein activity is an allosteric inhibitor of Src or TBK1 protein activity;
(iv) an inhibitor or depleter of NFKB or IRF3 protein or an inhibitor of RalB protein activation,
wherein the inhibitor of NFKB or IRF3 protein activity is an allosteric inhibitor of NFKB or IRF3 protein activity; or
(v) any combination of (i) to (iv); and

(2) at least one Growth Factor Inhibitor;

wherein optionally the cell is a tumor cell, a cancer cell, a cancer stem cell, or a dysfunctional cell.

The details of one or more embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference for all purposes.

### BRIEF DESCRIPTION OF THE DRAWINGS

The drawings set forth herein are illustrative of embodiments of the invention and are not meant to limit the scope of the invention as encompassed by the claims.

FIG. 1A schematically illustrates how Integrin αᵥβ₃ activates CRAF kinase, an enzyme promoting the growth and survival of human cancer cells, and FIG. 1B illustrates identification of a serine 338 (Ser 338) phosphorylation of a c-RAF by immunoprecipitation analysis.

FIG. 2 schematically illustrates how cilengitide blocks C-RAF S338 phosphorylation in brain tumors and is a biomarker of disease progress and is a surrogate marker of drug activity; FIG. 2A illustrates the study protocol, FIG. 2B graphically illustrates that cilengitide decrease relative tumor volume, FIGS. 2C and 2D illustrate untreated and cilengitide treated animals, respectively, and that cilengitide treatment blocks C-RAF S338 phosphorylation in brain tumors.

FIG. 3 illustrates how integrins and the extracellular matrix support the growth and malignancy of tumors; and that heterodimeric cell surface receptors that consist of an integrin a and b (α and β) subunit; and more than 24 distinct a-b heterodimers, and that the types of Integrins determine both the quality and quantity of the interactions between that cell and the extracellular matrix (ECM), and that integrin-mediated adhesion is required for cell responsiveness to most growth factors required for various biological processes including survival, migration, and invasion.

FIG. 4 illustrates the integrin family of adhesion proteins.

FIG. 5 schematically illustrates how integrins function on many cell types within the tumor microenvironment to regulate tumor progression.

FIG. 6 illustrates how integrins promote adhesion-dependent signaling; FIG. 5A illustrates biological effects of integrins on tumor cells; FIG. 6B illustrates cell staining with anti-FAK antibody to illustrate integrin heterodimeric cell surface receptor binding to extracellular matrix protein (ECM) via the “RGD” epitope, as schematically illustrated in FIG. 6C.

FIG. 7 schematically illustrates how a soluble “RGD” epitope can inhibit integrin binding to extracellular matrix protein (ECM) and integrin signaling.

FIG. 8A schematically illustrates how integrins mediate adhesion-dependent signaling telling a cell to proliferate, invade, differentiate or die, and binding and signaling molecules involved, and FIG. 8B illustrates cell staining with anti-FAK antibody to illustrate integrin heterodimeric cell surface receptor binding to extracellular matrix protein (ECM).

FIG. 9 illustrates how integrin aᵥb₃ is found on tumor but not normal vessels, with FIG. 9A showing tumor-adjacent normal tissue and FIG. 9B showing breast cancer tumor vessel cells.

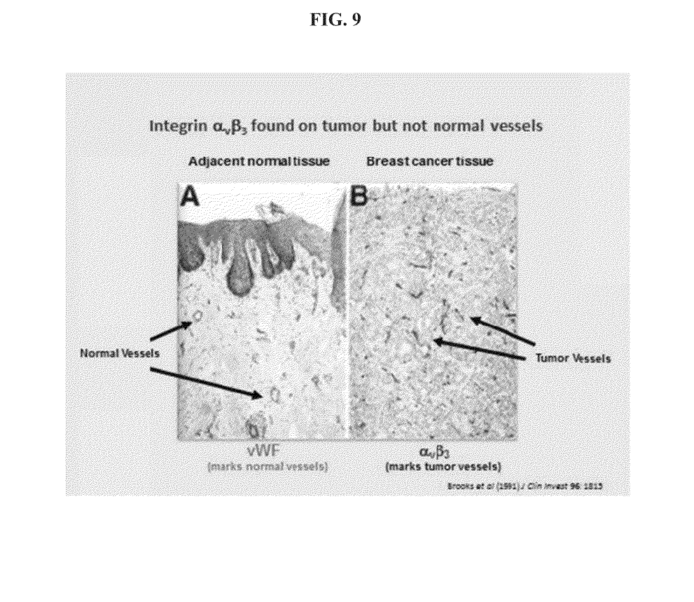

FIG. 10 illustrates how integrin aᵥb₃ is required for angiogenesis and tumor growth, with FIG. 10A showing that antagonizing integrin aᵥb₃ function with a cyclic RGD peptide or antibody disrupts the tumor vasculature in various animal models of human cancer; and FIG. 10B graphically illustrates tumor weight for treatment and control, and that that this anti-angiogenic effect impedes the growth of several tumor types including melanoma, pancreatic, lung, and laryngeal carcinomas.

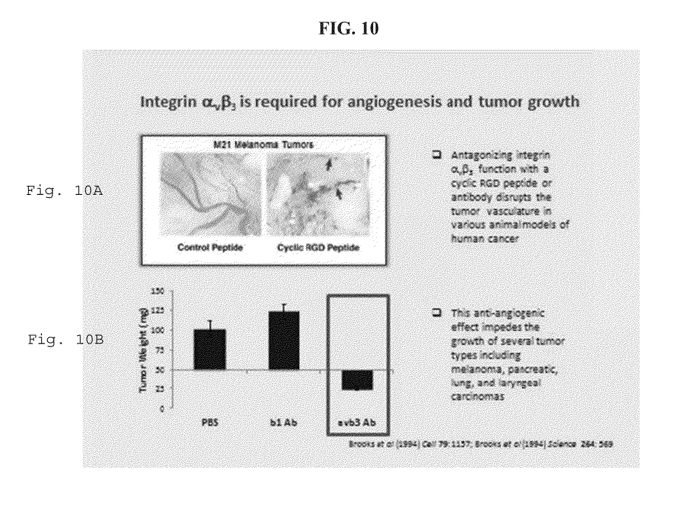

FIG. 11 describes how the expression of integrin aᵥb₃ on certain tumors correlates with tumor progression and metastasis.

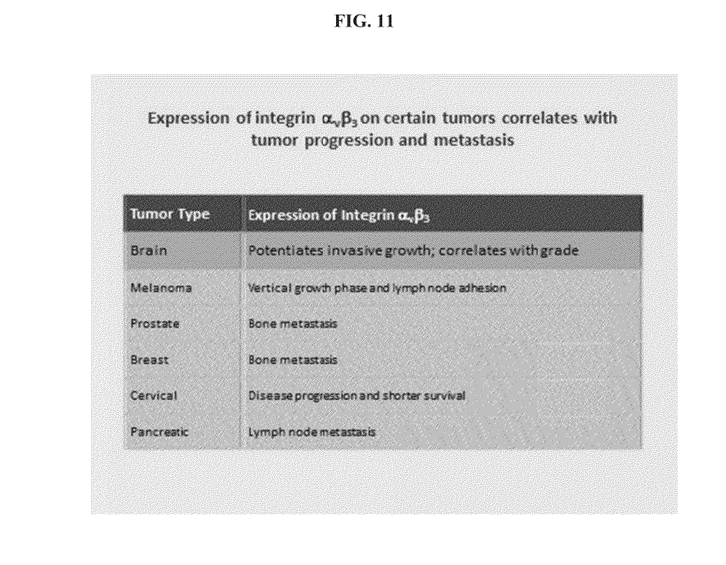

FIG. 12 illustrates how the adhesion protein vitronectin and its receptor, integrin aᵥb₃ are expressed in the most aggressive GBM tumors; FIG. 12A illustrates a tissue section showing vitronectin staining (brown) in a glioblastoma (GBM) biopsy (arrowheads indicate tumor border); FIG. 12B graphically illustrates relatively amounts of expression of integrin aᵥb₃ and vitronectin in GBM, showing that vitronectin, an extracellular matrix protein and integrin aᵥb₃ ligand, is expressed in GBM but NOT in normal brain tissue, and that integrin aᵥb₃ expression in both GBM cells and vasculature correlates with tumor grade.

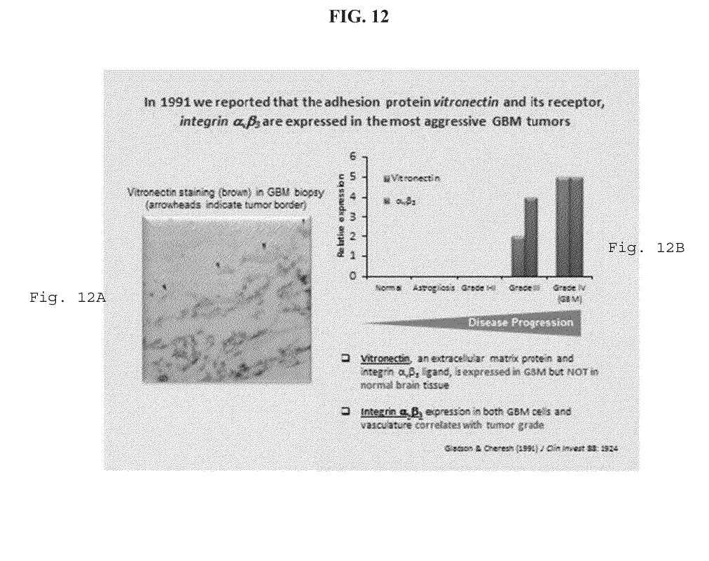

FIG. 13 illustrates how aᵥb₃ inhibitors that are potent, selective and safe were identified; where a library of cyclic peptides was screened in a cell-free integrin binding assay using aᵥb₃, and a_{IIb}b₃(platelet integrin), and one compound that looked promising, EMD 121974, proved to be a potent inhibitor of aᵥb₃ and aᵥb₅ yet did not inhibit the platelet integrin a_{IIb}b₃ associated with clotting; and that it was demonstrated that this agent inhibited angiogenesis and tumor growth in vivo, and following PK and safety studies, cancer patient studies were initiated in various patients with cancer and the drug cilengitide was shown to be non-toxic and potentially efficacious in some patients.

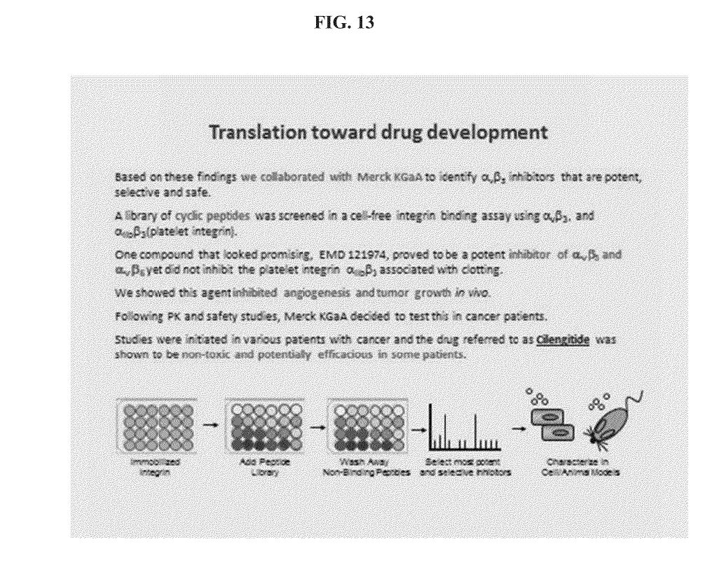

FIG. 14A illustrates the structure of cilengitide, and how it was selected from a screen of cyclic peptides for its capacity to inhibit ligand binding to integrins aᵥb₃ and aᵥb₅, but not a_{IIb}b₃. FIG. 14B schematically illustrates that cilengitide is thought to act in part, through the inhibition of tumor blood vessel growth, and that its effect on the tumor cells directly is likely a major contributor to its antitumor activity.

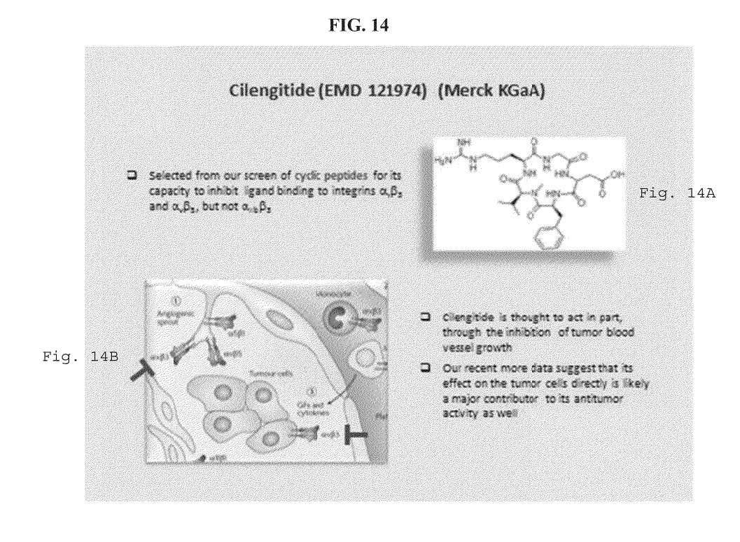

FIG. 15 illustrates that cilengitide inhibits brain tumor growth in mice in a context-specific manner; FIG. 15A illustrates the study protocol; and FIG. 15B graphically illustrates tumor size relative to control in brain versus (vs) skin, and that cilengitide decreases tumor burden in orthotopic brain tumor mouse model, and cilengitide inhibits the growth GBM tumors implanted in the brain but not in the skin of the same animal, and it suggests that brain microenvironment is a major determinant of tumor dependence on aᵥb₃.

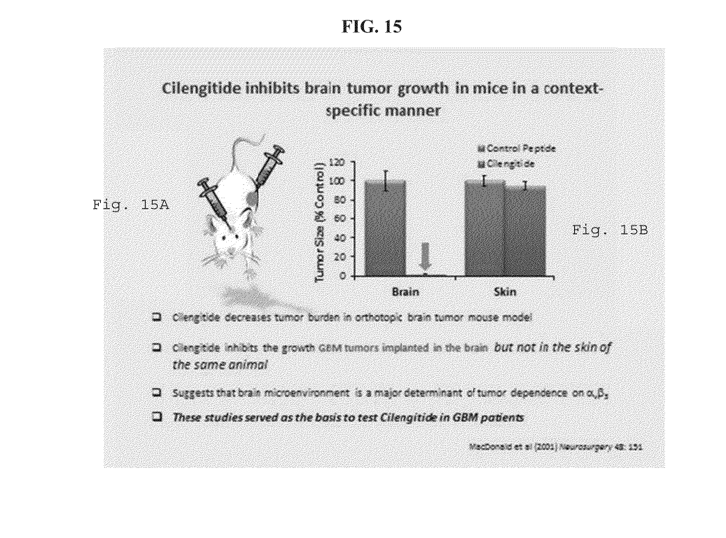

FIG. 16 illustrates that treatment with cilengitide inhibits orthotopic GBM tumor vascularization and growth in vivo, showing blood vessel, proliferating and dying cells stained with CD 31 (Platelet endothelial cell adhesion molecule (PECAM-1)), Ki-67 (a nuclear protein that is associated with and may be necessary for cellular proliferation) and TUNEL (Terminal deoxynucleotidyl transferase dUTP nick end labeling).

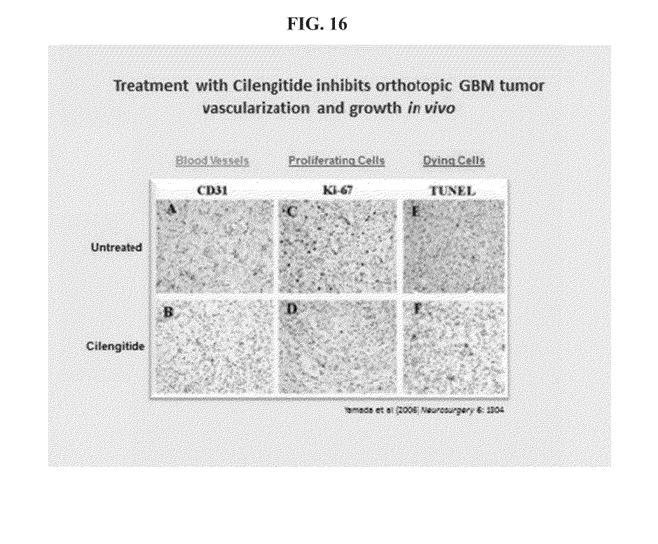

FIG. 17 shows that although originally intended as an anti-angiogenic therapy, cilengitide can inhibit aᵥ integrins on multiple cell types within a given tumor, including the tumor cells themselves, where FIG. 17A and FIG. 17B illustrate aᵥ integrin staining on tumor vessels and tumor cells, respectively.

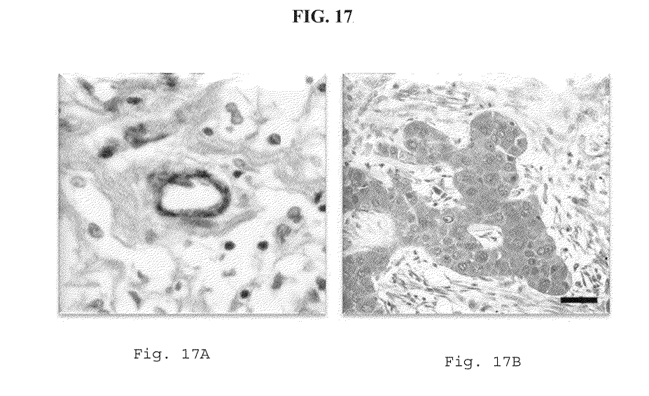

FIG. 18 schematically illustrates how integrin aᵥb₃ regulates RAF kinase, an enzyme promoting the growth and survival of human GBM cells, and that RAS-RAF signaling axis plays a central role in the progression of several cancer types including GBM, and integrin aᵥb₃ activates RAF and thus promotes cell survival and tumor angiogenesis, and that the inventors uncovered an unexpected role for RAF in promoting malignant cell cycle progression.

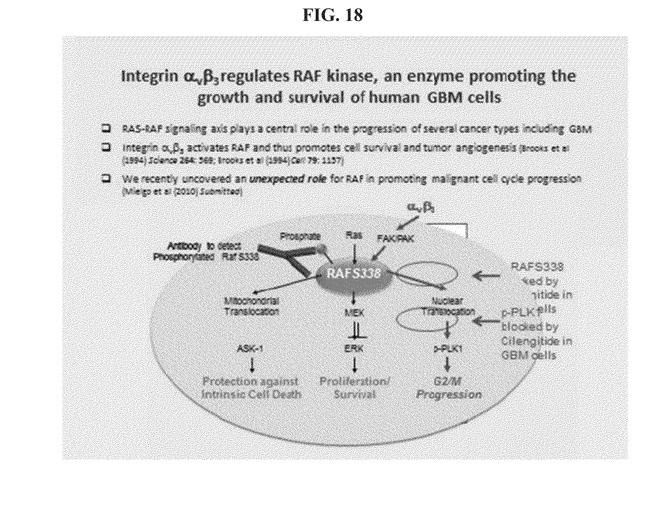

FIG. 19 illustrates how cilengitide blocks C-RAF S338 phosphorylation in brain tumors, and how C-RAF S338 phosphorylation is a biomarker of disease progress and surrogate marker of drug activity; FIG. 19A illustrates the study protocol; FIG. 19B illustrates how administration of cilengitide lowers relative tumor volume, and FIG. 19C illustrates histologic sections of brains treated with cilengitide (and control) and that cilengitide blocks C-RAF S338 phosphorylation in brain tumors.

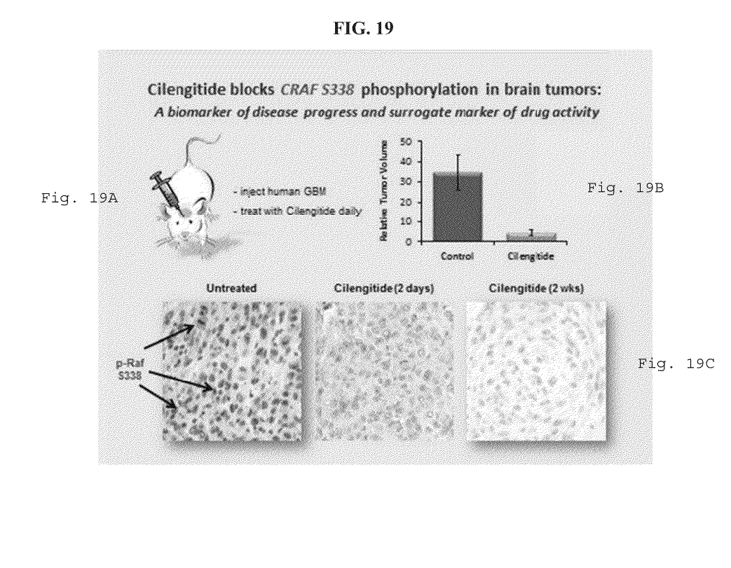

FIG. 20 illustrates that a phospho-mimetic C-RAF Serine 338 (S338D) mutation can drive orthotopic brain tumor growth, FIG. 20A illustrating a staining of orthotopic brain tumors, showing that phospho-mimetic C-RAF Serine 338 (S338D) mutation drives brain tumor growth, and FIG. 20B graphically illustrating this.

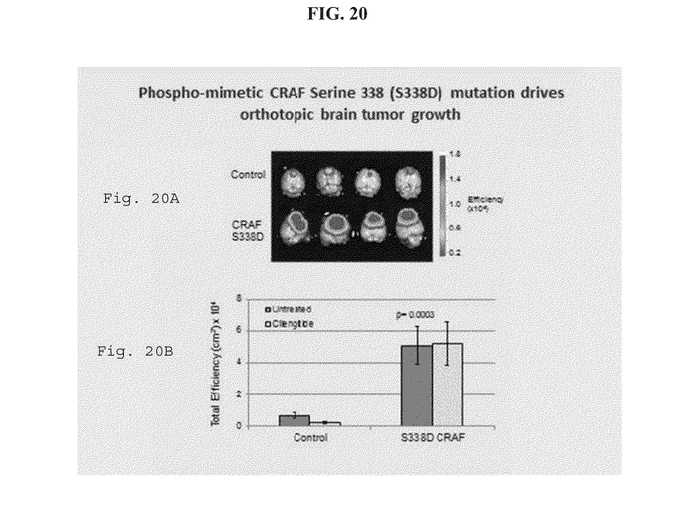

FIG. 21 illustrates that cilengitide treatment blocks G2/M progression; FIG. 21A illustrates the biological pathway of cilengitide blocking of G2/M progression; FIG. 21B illustrates a cell stain indicating cells are blocked in G2/M after cilengitide treatment, and FIG. 21C graphically illustrates this.

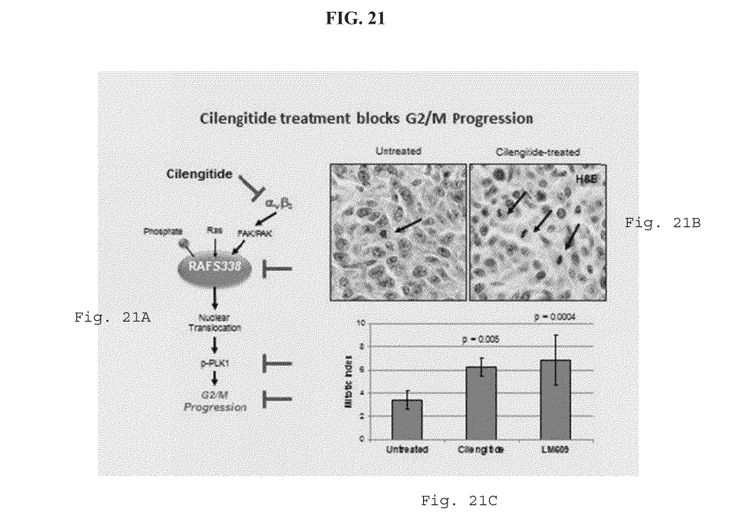

FIG. 22 illustrates that sub-optimal doses of cilengitide and radiation synergize to reduce orthotopic GBM tumor burden, with FIG. 22A illustrating the protocol of the study and FIG. 22B graphically illustrating the results.

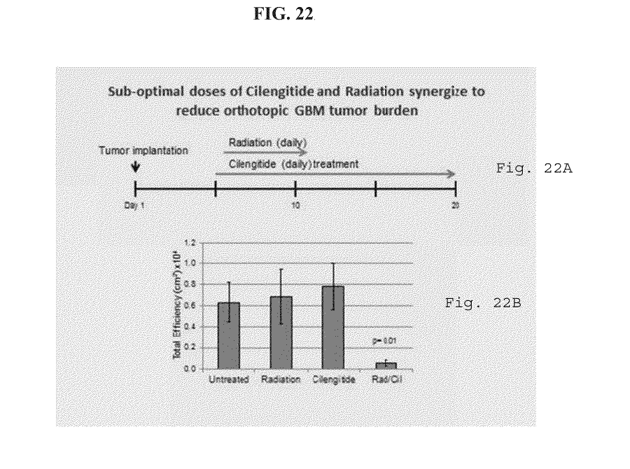

FIG. 23A graphically illustrates data showing that PAK activity is required for integrin avb3-mediated CRAF S338 activation: Western blot analysis of CRAF S338 phosphorylation status (p-CRAF S338) in serum-starved U87MG and U373 human glioma cells, expressing dominant-negative (DN) FAK or PAK, following ligation of integrin avb3 to vitronectin (VN) or b1 integrins to collagen (COL). FIG. 23B schematically illustrates a possibly pathway of action.

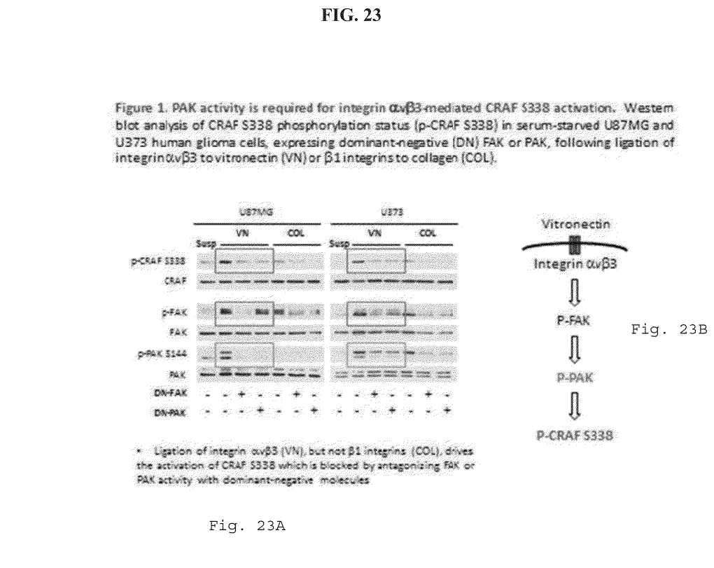

FIG. 24 illustrates data showing that pharmacologic inhibition of PAK blocks CRAF S338 activation. FIG. 24A schematically illustrates the design of the study giving the results of FIG. 24B, which illustrates Western blot analysis of CRAF S338 and PAK family member phosphorylation status in serum-starved human glioma cells following ligation of integrin avb3 to vitronectin in the presence of various doses of a PAK inhibitor (PAKi); the data demonstrates that the Afraxis (La Jolla, Calif.) PAK inhibitor blocks PAK1, PAK2, PAK4, and CRAF S338 activation, following ligation of integrin avb3 to vitronectin, in a dose-dependent manner.

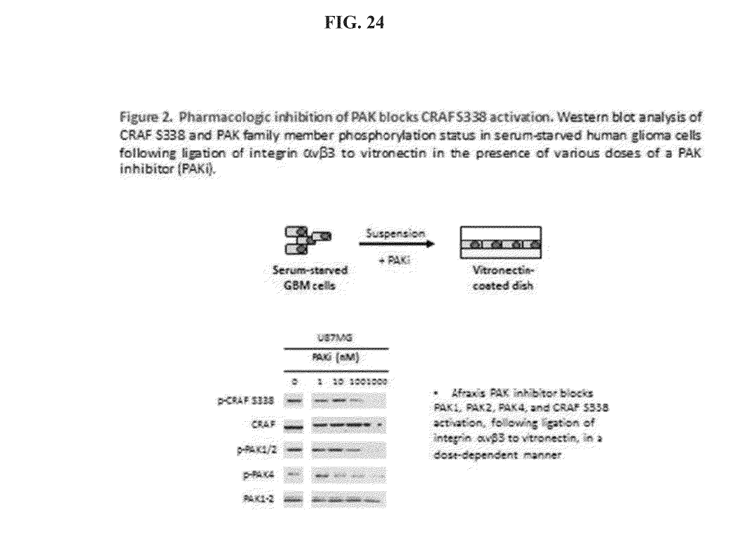

FIG. 25A and FIG. 25B schematically illustrate FACS analysis data showing that pharmacologic inhibition of PAK causes the accumulation of cells in G2/M: FACS analysis of cell cycle phases in serum-starved human U87MG glioma cells grown on vitronectin-coated plates in the presence a PAK inhibitor (PAKi) overnight. FIG. 25C graphically illustrates data from FIG. 25A and FIG. 25B showing that inhibition of PAK causes a robust increase in the number of cells in the G2-phase of the cell cycle→suggesting of a G2/M arrest with a concomitant decrease in the number of cells in the G1-phase.

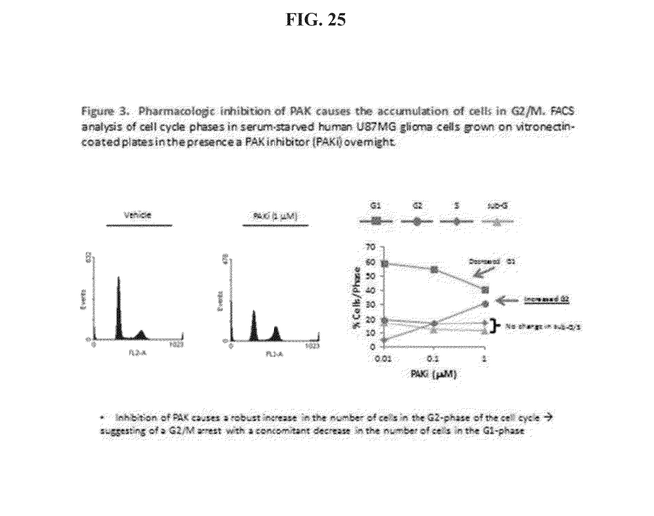

FIG. 26 illustrates that integrin αvβ3 expression promotes resistance to EGFR TKI: FIG. 26(a) illustrates flow cytometric quantification of cell surface markers after 3 weeks treatment with erlotinib (pancreatic and colon cancer cells) or lapatinib (breast cancer cells); FIG. 26(b) illustrates flow cytometric analysis of αvβ3 expression in FG and Miapaca-2 cells following erlotinib; FIG. 26 (c) illustrates: Top, immunofluorescence staining of integrin αᵥβ3 in tissue specimens obtained from orthotopic pancreatic tumors treated with vehicle or erlotinib; Bottom, Integrin αvβ3 expression was quantified as ratio of integrin αvβ3 pixel area over nuclei pixel area using Metamorph; FIG. 26(d) Right, intensity of β3 expression in mouse orthotopic lung tumors treated with vehicle or erlotinib, Left, immunohistochemical staining of 133, FIG. 26(f) illustrates tumor sphere formation assay to establish a dose-response for erlotinib, FIG. 26(g) illustrates orthotopic FG tumors treated for 10 days with vehicle or erlotinib, results are expressed as % tumor weight compared to vehicle control, immunoblot analysis for tumor lysates after 10 days of erlotinib confirms suppressed EGFR phosphorylation; as discussed in detail in Example 1, below.

FIG. 27 illustrates that integrin αvβ3 cooperates with K-RAS to promote resistance to EGFR blockade: FIG. 27(a-b) illustrates tumor sphere formation assay of FG expressing (a) or lacking (b) integrin β3 depleted of KRAS (shKRAS) or not (shCTRL) and treated with a dose response of erlotinib; FIG. 27(c) illustrates confocal microscopy images of PANC-1 and FG-β3 cells grown in suspension; FIG. 27(d) illustrates RAS activity assay performed in PANC-1 cells using GST-Raft-RBD immunoprecipitation as described below; as discussed in detail in Example 1, below.

FIG. 28 illustrates that RalB is a key modulator of integrin αvβ3-mediated EGFR TKI resistance: FIG. 28(a) illustrates tumor spheres formation assay of FG-β3 treated with non-silencing (shCTRL) or RalB-specific shRNA and exposed to a dose response of erlotinib; FIG. 28(b) illustrates effects of depletion of RalB on erlotinib sensitivity in β3-positive tumor in a pancreatic orthotopic tumor model; FIG. 28(c) illustrates tumor spheres formation assay of FG cells ectopically expressing vector control, WT RalB FLAG tagged constructs or a constitutively active RalB G23V FLAG tagged treated with erlotinib (0.5 μM); FIG. 28(d) illustrates RalB activity was determined in FG, FG-β3 expressing non-silencing or KRAS-specific shRNA, by using a GST-RalBP1-RBD immunoprecipitation assay; FIG. 28(e) illustrates: Right, overall active Ral immunohistochemical staining intensity between P3 negative and f33 positive human tumors; as discussed in detail in Example 1, below.

FIG. 29 illustrates that integrin αvβ3/RalB complex leads to NF-μB activation and resistance to EGFR TKI: FIG. 29(a) illustrates an immunoblot analysis of FG, FG-β3 and FG-β3 stably expressing non-silencing or RalB-specific ShRNA, grown in suspension and treated with erlotinib (0.5 μM); FIG. 29(b) illustrates tumor spheres formation assay of FG cells ectopically expressing vector control, WT NF-κB FLAG tagged or constitutively active S276D NF-κB FLAG tagged constructs treated with erlotinib; FIG. 29(c) illustrates tumor spheres formation assay of FG-β3 treating with non-silencing (shCTRL) or NF-κB-specific shRNA and exposed to erlotinib; FIG. 29(d) illustrates dose response in FG-β3 cells treated with erlotinib (10 nM to 5 μM), lenalidomide (10 nM to 5 μM) or a combination of erlotinib (10 nM to 5 μM) and lenalidomide (1 μM); FIG. 29(e) illustrates Model depicting the integrin αvβ3-mediated EGFR TKI resistance and conquering EGFR TKI resistance pathway and its downstream RalB and NF-κB effectors; as discussed in detail in Example 1, below.

FIG. 30 (Supplementary FIG. 1) illustrates that prolonged exposure to erlotinib induces Integrin αᵥβ₃ expression in lung tumors; representative immunohistochemical staining of integrin β3 in mouse tissues obtained from H441 orthotopic lung tumors long-term treated with either vehicle or erlotinib (scale bar, 100 μm); as discussed in detail in Example 1, below.

FIG. 31 (Supplementary FIG. 2) illustrates integrin αᵥβ3, even in its unligated state, promotes resistance to Growth Factor inhibitors but not to chemotherapies: FIG. 31(a) illustrates a tumor sphere formation assay comparing FG lacking β3 (FG), FG expressing β3 wild type (FG-β3) or the β3 D119A (FG-D119A) ligand binding domain mutant, treated with a dose response of erlotinib (Error bars represent s.d. (n=3 independent experiments); FIG. 31(b) illustrates tumor sphere formation assay of FG and FG-β3 cells untreated or treated with erlotinib (0.5 μM), OSI-906 (0.1 μM), gemcitabine (0.01 μM) or cisplatin (0.1 μM); FIG. 31(c) illustrates the effect of dose response of indicated treatments on tumor sphere formation (Error bars represent s.d. (n=3 independent experiments); as discussed in detail in Example 1, below.

FIG. 32 (Supplementary FIG. 3) illustrates that integrin αvβ3 does not colocalize with active HRAS, NRAS and RRAS: FIG. 32(a) illustrates that Ras activity was determined in PANC-1 cells grown in suspension by using a GST-Raft-RBD immunoprecipitation assay as described in Methods, see Example 1 (data are representative of two independent experiments); FIG. 32(b) illustrates confocal microscopy images of PANC-1 cells grown in suspension and stained for KRAS, RRAS, HRAS, NRAS (red), integrin αvβ3 (green) and DNA (TOPRO-3, blue) (Scale bar, 10 μM. Data are representative of two independent experiments); as discussed in detail in Example 1, below.

FIG. 33(Supplementary FIG. 4) illustrates that Galectin-3 is required to promote integrin αvβ3/KRAS complex formation: FIG. 33(a-b) illustrates confocal microscopy images of Panc-1 cells lacking or expressing integrin αvβ3 grown in suspension; FIG. 33(a) illustrates cells stained for KRAS (green), Galectin-3 (red), and DNA (TOPRO-3, blue); FIG. 33(b) illustrates cells stained for integrin αvβ3 (green), Galectin-3 (red) and DNA (TOPRO-3, blue), Scale bar, 10 μm, data are representative of three independent experiments; FIG. 33(c) illustrates an immunoblot analysis of Galectin-3 immuno-precipitates from PANC-1 cells expressing non-silencing (sh CTRL) or integrin β3-specific shRNA (sh β3), data are representative of three independent experiments; FIG. 33(d) illustrates an immunoblot analysis of integrin β3 immunoprecipitates from PANC-1 cells expressing non-silencing (sh CTRL) or Galectin-3-specific shRNA (sh Gal3), data are representative of three independent experiments; as discussed in detail in Example 1, below.

FIG. 34 (Supplementary FIG. 5) illustrates that ERK, AKT and RalA are not specifically required to promote integrin αvβ3-mediated resistance to EGFR TKI; tumor spheres formation assay of FG and FG-β3 expressing non-silencing or ERK1/2, AKT1 and RalA-specific shRNA and treated with erlotinib (0.5 μM), error bars represent s.d. (n=3 independent experiments); as discussed in detail in Example 1, below.

FIG. 35 illustrates that RalB is sufficient to promote resistance to EGFR TKI: FIG. 35(a) (supplementary FIG. 6) illustrates a tumor sphere formation assay of FG expressing non-silencing or RalB specific shRNA and treated with a dose response of erlotinib. Error bars represent s.d. (n=3 independent experiments); FIG. 35(b) (supplementary FIG. 6) illustrates a tumor spheres formation assay of PANC-1 stably expressing integrin β3-specific shRNA and ectopically expressing vector control, WT RalB FLAG tagged or a constitutively active RalB G23V FLAG tagged constructs treated with erlotinib (0.5 μM), error bars represent s.d. (n=3 independent experiments); FIG. 35(c) (Supplementary FIG. 7) shows that integrin αvβ3 colocalizes with RalB in cancer cells: illustrates confocal microscopy images of Panc-1 cells grown in suspension. Cells are stained for integrin αvβ3 (green), RalB (red), pFAK (red), and DNA (TOPRO-3, blue), scale bar, 10 μm, data are representative of three independent experiments; as discussed in detail in Example 1, below.

FIG. 36 (Supplementary FIG. 8) illustrates that integrin αvβ3 colocalizes with RalB in human breast and pancreatic tumor biopsies and interacts with RalB in cancer cells: FIG. 36(a) illustrates confocal microscopy images of integrin αvβ3 (green), RalB (red) and DNA (TOPRO-3, blue) in tumor biopsies from breast and pancreatic cancer patients, Scale bar, 20 μm; FIG. 36(b) illustrates a Ral activity assay performed in PANC-1 cells using GST-RalBP1-RBD immunoprecipitation assay, Immunoblot analysis of RalB and integrin P3, data are representative of three independent experiments; as discussed in detail in Example 1, below.

Like reference symbols in the various drawings indicate like elements.

Reference will now be made in detail to various exemplary embodiments of the invention, examples of which are illustrated in the accompanying drawings. The following detailed description is provided to give the reader a better understanding of certain details of aspects and embodiments of the invention, and should not be interpreted as a limitation on the scope of the invention.

### DETAILED DESCRIPTION

### Methods for Using C-RAF Phosphorylation Status as a Surrogate Biomarker of Integrin Antagonist Activity in the Treatment of Human Cancers

In alternative embodiments, the invention provides compositions and methods for monitoring the activity of integrin antagonists, such as cilengitide (Merck KGaA, Darmstadt, Germany); or any small molecule, peptide, polypeptide or antibody that blocks activation of an alphaᵥ-beta₃ (or αᵥ-β₃) integrin polypeptide, or blocks the interaction of a ligand with an alphaᵥ-beta₃ (or αᵥ-β₃) integrin polypeptide, or blocks the phosphorylation of a C-RAF polypeptide, or blocks the phosphorylation of a C-RAF serine residue 338 (ser-338), in the treatment of a cancer, e.g., a human cancer, and identifying patient populations that will benefit from this therapy.

This invention for the first time finds that:
1) Phosphorylation of C-RAF represents the first surrogate biomarker for the efficacy of an integrin antagonist, such as cilengitide, in treating cancer patients;
2) Phosphorylation of C-RAF represents a predictive biomarker of tumor sensitivity to these agents.

In alternative embodiments, phosphorylation of (or the extent of phosphorylation of) a C-RAF serine residue 338 (ser-338) in a cell (e.g., from a biopsy, a smear, a scraping, or a cell, blood or serum sample) is a biomarker of disease (e.g., cancer, a metastasis) progress, and/or disease responsiveness to an integrin antagonist such as cilengitide, i.e., phosphorylation of (or the extent of phosphorylation of) a C-RAF serine residue 338 (ser-338) in a cell (e.g., from a biopsy) is a surrogate marker of drug activity.

While the invention is not limited by any particular mechanism of action, the monitoring (predictive) capabilities of the compositions and methods of the invention are based in a finding that inhibiting ligation of integrins with a small molecule or an antibody antagonist, such as Cilengitide (an RGD peptide), or LM609 (a monoclonal antibody), prevents the activation/phosphorylation of C-RAF Ser338 in both human glioblastoma cell lines and in a mouse orthotopic brain tumor model. These events are associated with reduced cell proliferation in vitro and tumor regression in vivo.

In alternative embodiments, confirmation that activation/phosphorylation of C-RAF Ser338 can serve as a biomarker of drug activity or identify individuals that would be responsive to a treatment (e.g., inhibition of activation/phosphorylation of C-RAF Ser338) can be by e.g., paraffin-embedded sections of tissue, e.g., biopsies, e.g., of tumors, e.g., of orthotopic brain tumors, treated with integrin antagonists, or not; these are being immunostained for activated C-RAF. In alternative embodiments, immuno-histochemical staining of phosphorylated C-RAF Ser338 in patient biopsies serves as both a prognostic and predictive biomarker in determining tumor sensitivity and response to integrin antagonists, such as cilengitide, pre- and post-treatment. Any protocol for immuno-histochemical staining of phosphorylated C-RAF Ser338 can be used, for example, anti-phospho-C-Raf (Ser388), anti-A-Raf, anti-B-Raf, anti-C-Raf, anti-phospho-MEK(Ser217/221), anti-MEK antibodies are all commercially available, e.g., Cell Signaling Technology, Inc. Danvers, Mass.

### Kits and Instructions

The invention provides kits comprising compositions for practicing the methods of the invention, including instructions for use thereof. In alternative embodiments, the invention provides kits comprising materials to determine the phosphorylation of C-RAF Ser338, e.g., anti-phospho-C-Raf (Ser388) antibodies. In alternative embodiments, the invention provides kits comprising a composition, product of manufacture, or mixture or culture of cells for practicing a method of the invention; wherein optionally the kit further comprises instructions for practicing a method of the invention, e.g., for identifying individuals that would be responsive to a treatment comprising (including) blocking activation of integrin polypeptide alphaᵥ-beta₃ (or αᵥ-β₃), or blocking the interaction of a ligand with integrin polypeptide alphaᵥ-beta₃ (or αᵥ-β₃).

### Compositions and Methods for Inhibiting PAKs and Cell Cycle Progression, and Treating Cancers

In alternative embodiments, the invention provides methods for directly inhibiting the activity or expression of a P21 protein (Cdc42/Rac)-Activated Kinase (PAK or c-PAK); or a human P21 protein (Cdc42/Rac)-Activated Kinase (PAK or c-PAK). While the invention is not limited by any particular mechanism of action, in alternative embodiments, in alternative embodiments the invention provides methods for: reducing or inhibiting serine 338 (Ser 338) phosphorylation of a c-RAF; reducing or inhibiting a dysfunctional cell, cancer cell or tumor growth such as a c-RAF-dependent dysfunctional cell, cancer cell or tumor growth; promoting a tumor regression in vivo in a human tumor or cancer cell such as a c-RAF-dependent human tumor or cancer cell; inducing double-stranded DNA breakage in a cell; and/or, sensitizing a tumor cell to a radiation (radiosensitizing a cell) or a chemotherapy.

### Pharmaceutical Compositions

In alternative embodiments, the invention provides pharmaceutical compositions for practicing the methods of the invention, e.g., pharmaceutical compositions for reducing or inhibiting a dysfunctional cell, cancer cell or tumor growth; or, for inducing double-stranded DNA breakage in a cell; or, for sensitizing a tumor cell to a radiation (radiosensitizing a cell) or a chemotherapy. The invention provides compositions as described herein, including pharmaceutical compositions, e.g., in the manufacture of medicaments for ameliorating, preventing and/or treating diseases, infections and/or conditions having unwanted, pathological or aberrant cell proliferation, or, for sensitizing a tumor cell to a radiation (radiosensitizing a cell) or a chemotherapy.

In alternative embodiments, compositions used to practice the methods of the invention are formulated with a pharmaceutically acceptable carrier. In alternative embodiments, the pharmaceutical compositions used to practice the methods of the invention can be administered parenterally, topically, orally or by local administration, such as by aerosol or transdermally. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration are well described in the scientific and patent literature, see, e.g., the latest edition of Remington's Pharmaceutical Sciences, Maack Publishing Co, Easton Pa. (“Remington's”).

Therapeutic agents used to practice the methods of the invention can be administered alone or as a component of a pharmaceutical formulation (composition). The compounds may be formulated for administration in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the compositions used to practice the methods of the invention include those suitable for oral/nasal, topical, parenteral, rectal, and/or intravaginal administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect.

Pharmaceutical formulations used to practice the methods of the invention can be prepared according to any method known to the art for the manufacture of pharmaceuticals. Such drugs can contain sweetening agents, flavoring agents, coloring agents and preserving agents. A formulation can be admixtured with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

Pharmaceutical formulations for oral administration can be formulated using pharmaceutically acceptable carriers well known in the art in appropriate and suitable dosages. Such carriers enable the pharmaceuticals to be formulated in unit dosage forms as tablets, geltabs, pills, powder, dragees, capsules, liquids, lozenges, gels, syrups, slurries, suspensions, etc., suitable for ingestion by the patient. Pharmaceutical preparations for oral use can be formulated as a solid excipient, optionally grinding a resulting mixture, and processing the mixture of granules, after adding suitable additional compounds, if desired, to obtain tablets or dragee cores. Suitable solid excipients are carbohydrate or protein fillers include, e.g., sugars, including lactose, sucrose, mannitol, or sorbitol; starch from corn, wheat, rice, potato, or other plants; cellulose such as methyl cellulose, hydroxypropylmethyl-cellulose, or sodium carboxy-methylcellulose; and gums including arabic and tragacanth; and proteins, e.g., gelatin and collagen. Disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations used to practice the methods of the invention can also be used orally using, e.g., push-fit capsules made of gelatin, as well as soft, sealed capsules made of gelatin and a coating such as glycerol or sorbitol. Push-fit capsules can contain active agents mixed with a filler or binders such as lactose or starches, lubricants such as talc or magnesium stearate, and, optionally, stabilizers. In soft capsules, the active agents can be dissolved or suspended in suitable liquids, such as fatty oils, liquid paraffin, or liquid polyethylene glycol with or without stabilizers.

Aqueous suspensions can contain an active agent (e.g., a composition used to practice a method of the invention) in admixture with excipients suitable for the manufacture of aqueous suspensions. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Oil-based pharmaceuticals can be useful for administration of hydrophobic active agents used to practice a method of the invention. Oil-based suspensions can be formulated by suspending an active agent in a vegetable oil, such as arachis oil, olive oil, sesame oil or coconut oil, or in a mineral oil such as liquid paraffin; or a mixture of these. See e.g., U.S. Pat. No. 5,716,928 describing using essential oils or essential oil components for increasing bioavailability and reducing inter- and intra-individual variability of orally administered hydrophobic pharmaceutical compounds (see also U.S. Pat. No. 5,858,401). The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto (1997) J. Pharmacol. Exp. Ther. 281:93-102. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

In practicing this invention, the pharmaceutical compounds can also be administered by in intranasal, intraocular and intravaginal routes including suppositories, insufflation, powders and aerosol formulations (for examples of steroid inhalants, see Rohatagi (1995) J. Clin. Pharmacol. 35:1187-1193; Tjwa (1995) Ann. Allergy Asthma Immunol. 75:107-111). Suppositories formulations can be prepared by mixing the drug with a suitable non-irritating excipient which is solid at ordinary temperatures but liquid at body temperatures and will therefore melt in the body to release the drug. Such materials are cocoa butter and polyethylene glycols.

In practicing this invention, the pharmaceutical compounds can be delivered by transdermally, by a topical route, formulated as applicator sticks, solutions, suspensions, emulsions, gels, creams, ointments, pastes, jellies, paints, powders, and aerosols.

In practicing this invention, the pharmaceutical compounds can also be delivered as microspheres for slow release in the body. For example, microspheres can be administered via intradermal injection of drug which slowly release subcutaneously; see Rao (1995) J. Biomater Sci. Polym. Ed. 7:623-645; as biodegradable and injectable gel formulations, see, e.g., Gao (1995) Pharm. Res. 12:857-863 (1995); or, as microspheres for oral administration, see, e.g., Eyles (1997) J. Pharm. Pharmacol. 49:669-674.

In practicing this invention, the pharmaceutical compounds can be parenterally administered, such as by intravenous (IV) administration or administration into a body cavity or lumen of an organ. These formulations can comprise a solution of active agent dissolved in a pharmaceutically acceptable carrier. Acceptable vehicles and solvents that can be employed are water and Ringer's solution, an isotonic sodium chloride. In addition, sterile fixed oils can be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally-acceptable diluent or solvent, such as a solution of 1,3-butanediol. The administration can be by bolus or continuous infusion (e.g., substantially uninterrupted introduction into a blood vessel for a specified period of time).

The pharmaceutical compounds and formulations used to practice a method of the invention can be lyophilized. The invention provides a stable lyophilized formulation comprising a composition of the invention, which can be made by lyophilizing a solution comprising a pharmaceutical of the invention and a bulking agent, e.g., mannitol, trehalose, raffinose, and sucrose or mixtures thereof. A process for preparing a stable lyophilized formulation can include lyophilizing a solution about 2.5 mg/mL protein, about 15 mg/mL sucrose, about 19 mg/mL NaCl, and a sodium citrate buffer having a pH greater than 5.5 but less than 6.5. See, e.g., U.S. patent app. no. 20040028670.

The compositions and formulations used to practice the methods of the invention can be delivered by the use of liposomes (see also discussion, below). By using liposomes, particularly where the liposome surface carries ligands specific for target cells, or are otherwise preferentially directed to a specific organ, one can focus the delivery of the active agent into target cells in vivo. See, e.g., U.S. Pat. Nos. 6,063,400; 6,007,839; Al-Muhammed (1996) J. Microencapsul. 13:293-306; Chonn (1995) Curr. Opin. Biotechnol. 6:698-708; Ostro (1989) Am. J. Hosp. Pharm. 46:1576-1587.

The formulations used to practice the methods of the invention can be administered for prophylactic and/or therapeutic treatments.

In alternative embodiments of therapeutic applications and methods of the invention, compositions are administered to a subject or patient already suffering from a condition, infection or disease (e.g., a cancer) in an amount sufficient to cure, alleviate or partially arrest the clinical manifestations of the condition, infection or disease and its complications (a “therapeutically effective amount”). For example, in alternative embodiments, pharmaceutical compositions of the invention are administered in an amount sufficient to treat, prevent and/or ameliorate normal, dysfunction (e.g., abnormally proliferating) cell, e.g., cancer cell, or blood vessel cell, including endothelial and/or capillary cell growth; including neovasculature related to (within, providing a blood supply to) hyperplastic tissue, a granuloma or a tumor. In alternative embodiments, pharmaceutical compositions of the invention are administered in an amount sufficient to radiosensitize a cancer cell, a cancer stem cell, or a tumor. The amount of pharmaceutical composition adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods of the invention are correct and appropriate.

Single or multiple administrations of formulations can be given depending on the dosage and frequency as required and tolerated by the patient. The formulations should provide a sufficient quantity of active agent to effectively treat, prevent or ameliorate a conditions, diseases or symptoms as described herein. For example, an exemplary pharmaceutical formulation for oral administration of compositions used to practice the methods of the invention can be in a daily amount of between about 0.1 to 0.5 to about 20, 50, 100 or 1000 or more ug per kilogram of body weight per day. In an alternative embodiment, dosages are from about 1 mg to about 4 mg per kg of body weight per patient per day are used. Lower dosages can be used, in contrast to administration orally, into the blood stream, into a body cavity or into a lumen of an organ. Substantially higher dosages can be used in topical or oral administration or administering by powders, spray or inhalation. Actual methods for preparing parenterally or non-parenterally administrable formulations will be known or apparent to those skilled in the art and are described in more detail in such publications as Remington's, supra.

The methods of the invention can further comprise co-administration with other drugs or pharmaceuticals, e.g., with other radiosensitizing agents such as misonidazole, metronidazole and/or hypoxic cytotoxins such as tirapazamine.

In alternative embodiments, the methods and/or compositions and formulations of the invention can be co-formulated with and/or co-administered with antibiotics (e.g., antibacterial or bacteriostatic peptides or proteins), particularly those effective against gram negative bacteria, fluids, cytokines, immunoregulatory agents, anti-inflammatory agents, complement activating agents, such as peptides or proteins comprising collagen-like domains or fibrinogen-like domains (e.g., a ficolin), carbohydrate-binding domains, and the like and combinations thereof.

### Nanoparticles and Liposomes

The invention also provides nanoparticles and liposomal membranes comprising compounds used to practice the methods of the invention which target specific molecules, including biologic molecules, such as polypeptide, including cell surface polypeptides, e.g., polypeptides on abnormally growing cells, cancer cells, cancer stem cells, blood vessel and angiogenic cells. Thus, in alternative embodiments; the invention provides nanoparticles and liposomal membranes targeting diseased and/or tumor (cancer) stem cells and dysfunctional stem cells, and angiogenic cells.

In alternative embodiments, the invention provides nanoparticles and liposomal membranes comprising (in addition to comprising compounds used to practice the methods of the invention) molecules, e.g., peptides or antibodies, that selectively target abnormally growing, diseased, infected, dysfunctional and/or cancer (tumor) cell receptors. In alternative embodiments, the invention provides nanoparticles and liposomal membranes using IL-11 receptor and/or the GRP78 receptor to targeted receptors on cells, e.g., on tumor cells, e.g., on prostate or ovarian cancer cells. See, e.g., U.S. patent application publication no. 20060239968.

In one aspect, the compositions used to practice the methods of the invention are specifically targeted for inhibiting, ameliorating and/or preventing endothelial cell migration and for inhibiting angiogenesis, e.g., tumor-associated or disease- or infection-associated neovasculature.

The invention also provides nanocells to allow the sequential delivery of two different therapeutic agents with different modes of action or different pharmacokinetics, at least one of which comprises a composition used to practice the methods of the invention. A nanocell is formed by encapsulating a nanocore with a first agent inside a lipid vesicle containing a second agent; see, e.g., Sengupta, et al., U.S. Pat. Pub. No. 20050266067. The agent in the outer lipid compartment is released first and may exert its effect before the agent in the nanocore is released. The nanocell delivery system may be formulated in any pharmaceutical composition for delivery to patients suffering from a diseases or condition as described herein, e.g., such as a retinal age-related macular degeneration, a diabetic retinopathy, a cancer or carcinoma, a glioblastoma, a neuroma, a neuroblastoma, a colon carcinoma, a hemangioma, an infection and/or a condition with at least one inflammatory component, and/or any infectious or inflammatory disease, such as a rheumatoid arthritis, a psoriasis, a fibrosis, leprosy, multiple sclerosis, inflammatory bowel disease, or ulcerative colitis or Crohn's disease.

In treating cancer, a traditional antineoplastic agent is contained in the outer lipid vesicle of the nanocell, and an antiangiogenic agent of this invention is loaded into the nanocore. This arrangement allows the antineoplastic agent to be released first and delivered to the tumor before the tumor's blood supply is cut off by the composition of this invention.

The invention also provides multilayered liposomes comprising compounds used to practice this invention, e.g., for transdermal absorption, e.g., as described in Park, et al., U.S. Pat. Pub. No. 20070082042. The multilayered liposomes can be prepared using a mixture of oil-phase components comprising squalane, sterols, ceramides, neutral lipids or oils, fatty acids and lecithins, to about 200 to 5000 nm in particle size, to entrap a composition of this invention.

A multilayered liposome used to practice the invention may further include an antiseptic, an antioxidant, a stabilizer, a thickener, and the like to improve stability. Synthetic and natural antiseptics can be used, e.g., in an amount of 0.01% to 20%. Antioxidants can be used, e.g., BHT, erysorbate, tocopherol, astaxanthin, vegetable flavonoid, and derivatives thereof, or a plant-derived antioxidizing substance. A stabilizer can be used to stabilize liposome structure, e.g., polyols and sugars. Exemplary polyols include butylene glycol, polyethylene glycol, propylene glycol, dipropylene glycol and ethyl carbitol; examples of sugars are trehalose, sucrose, mannitol, sorbitol and chitosan, or a monosaccharides or an oligosaccharides, or a high molecular weight starch. A thickener can be used for improving the dispersion stability of constructed liposomes in water, e.g., a natural thickener or an acrylamide, or a synthetic polymeric thickener. Exemplary thickeners include natural polymers, such as acacia gum, xanthan gum, gellan gum, locust bean gum and starch, cellulose derivatives, such as hydroxy ethylcellulose, hydroxypropyl cellulose and carboxymethyl cellulose, synthetic polymers, such as polyacrylic acid, poly-acrylamide or polyvinylpyrollidone and polyvinylalcohol, and copolymers thereof or cross-linked materials.

Liposomes can be made using any method, e.g., as described in Park, et al., U.S. Pat. Pub. No. 20070042031, including method of producing a liposome by encapsulating a therapeutic product comprising providing an aqueous solution in a first reservoir; providing an organic lipid solution in a seqond reservoir, wherein one of the aqueous solution and the organic lipid solution includes a therapeutic product; mixing the aqueous solution with said organic lipid solution in a first mixing region to produce a liposome solution, wherein the organic lipid solution mixes with said aqueous solution so as to substantially instantaneously produce a liposome encapsulating the therapeutic product; and immediately thereafter mixing the liposome solution with a buffer solution to produce a diluted liposome solution.

The invention also provides nanoparticles comprising compounds used to practice this invention to deliver a composition of the invention as a drug-containing nanoparticles (e.g., a secondary nanoparticle), as described, e.g., in U.S. Pat. Pub. No. 20070077286. In one embodiment, the invention provides nanoparticles comprising a fat-soluble drug of this invention or a fat-solubilized water-soluble drug to act with a bivalent or trivalent metal salt.

### Liposomes

The compositions and formulations used to practice the invention can be delivered by the use of liposomes. By using liposomes, particularly where the liposome surface carries ligands specific for target cells, or are otherwise preferentially directed to a specific organ, one can focus the delivery of the active agent into target cells in vivo. See, e.g., U.S. Pat. Nos. 6,063,400; 6,007,839; Al-Muhammed (1996) J. Microencapsul. 13:293-306; Chonn (1995) Curr. Opin. Biotechnol. 6:698-708; Ostro (1989) Am. J. Hosp. Pharm. 46:1576-1587. For example, in one embodiment, compositions and formulations used to practice the invention are delivered by the use of liposomes having rigid lipids having head groups and hydrophobic tails, e.g., as using a polyethyleneglycol-linked lipid having a side chain matching at least a portion the lipid, as described e.g., in US Pat App Pub No. 20080089928. In another embodiment, compositions and formulations used to practice the invention are delivered by the use of amphoteric liposomes comprising a mixture of lipids, e.g., a mixture comprising a cationic amphiphile, an anionic amphiphile and/or neutral amphiphiles, as described e.g., in US Pat App Pub No. 20080088046, or 20080031937. In another embodiment, compositions and formulations used to practice the invention are delivered by the use of liposomes comprising a polyalkylene glycol moiety bonded through a thioether group and an antibody also bonded through a thioether group to the liposome, as described e.g., in US Pat App Pub No. 20080014255. In another embodiment, compositions and formulations used to practice the invention are delivered by the use of liposomes comprising glycerides, glycerophospholipides, glycerophosphinolipids, glycerophosphonolipids, sulfolipids, sphingolipids, phospholipids, isoprenolides, steroids, stearines, sterols and/or carbohydrate containing lipids, as described e.g., in US Pat App Pub No. 20070148220.

Therapeutically Effective Amount and Dose

In alternative embodiment, pharmaceutical compositions and formulations used to practice the invention can be administered for prophylactic and/or therapeutic treatments; for example, the invention provides methods for treating, preventing or ameliorating: a disease or condition associated with dysfunctional stem cells or cancer stem cells, a retinal age-related macular degeneration, a diabetic retinopathy, a cancer or carcinoma, a glioblastoma, a neuroma, a neuroblastoma, a colon carcinoma, a hemangioma, an infection and/or a condition with at least one inflammatory component, and/or any infectious or inflammatory disease, such as a rheumatoid arthritis, a psoriasis, a fibrosis, leprosy, multiple sclerosis, inflammatory bowel disease, or ulcerative colitis or Crohn's disease. In therapeutic applications, compositions are administered to a subject already suffering from a condition, infection or disease in an amount sufficient to cure, alleviate or partially arrest the clinical manifestations of the condition, infection or disease (e.g., disease or condition associated with dysfunctional stem cells or cancer stem cells) and its complications (a “therapeutically effective amount”). In the methods of the invention, a pharmaceutical composition is administered in an amount sufficient to treat (e.g., ameliorate) or prevent a disease or condition associated with dysfunctional stem cells or cancer stem cells. The amount of pharmaceutical composition adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

### Kits and Instructions

The invention provides kits comprising compositions for practicing the methods of the invention, including instructions for use thereof. In alternative embodiments, the invention provides kits comprising a human P21 protein (Cdc42/Rac)-Activated Kinase (PAK or c-PAK) inhibitor. In alternative embodiments, the invention provides kits comprising a composition, product of manufacture, or mixture or culture of cells for practicing a method of the invention; wherein optionally the kit further comprises instructions for practicing a method of the invention.

### Compositions and Methods for Treating Diseases and Conditions Responsive to Cell Growth Inhibition by Growth Factor Inhibitors

In alternative embodiments, the invention provides compositions and methods for overcoming or diminishing or preventing Growth Factor Inhibitor (GFI) resistance in a cell, or, a method for increasing the growth-inhibiting effectiveness of a Growth Factor inhibitor on a cell, or, a method for re-sensitizing a cell to a Growth Factor Inhibitor (GFI). In alternative embodiments, the cell is a tumor cell, a cancer cell or a dysfunctional cell. In alternative embodiments, the invention provides compositions and methods for determining: whether an individual or a patient would benefit from or respond to administration of a Growth Factor Inhibitor, or, which individuals or patients would benefit from a combinatorial approach comprising administration of a combination of: at least one growth factor and at least one compound, composition or formulation used to practice a method of the invention, such as an NfKb inhibitor.

We found that integrin anb3 is upregulated in cells that become resistant to Growth Factor inhibitors. Our findings demonstrate that integrin anb3 promotes de novo and acquired resistance to Growth factor inhibitors by interacting and activating RalB. RalB activation leads to the activation of Src and TBK1 and the downstream effectors NFKB and IRF3. We also found that depletion of RalB or its downstream signaling (Src/NFKB) in b3-positive cells overcomes resistance to growth factor inhibitors. This invention demonstrates that the integrin anb3/RalB signaling complex promotes resistance to growth factor inhibitors; and in alternative embodiments, integrin αᵥβ₃ (anb3) and active RalB are used as biomarkers in patient samples to predict which patients will respond to growth factor inhibitors and which patients might rather benefit from alternative/combinatorial approaches such as a combination of growth factors and NfKb inhibitors.

This invention for the first time identifies integrin αvβ3 and active RalB as potential biomarker for tumors that are or have become (e.g., de novo and acquired) resistant to growth factors blockade. Accordingly, in alternative embodiments, the invention provides compositions and methods for the depletion of RalB, Src NFkB and its downstream signaling effectors to sensitize αvβ3-expressing tumors to growth factor blockade. These findings reveal a new role for integrin αvβ3 in mediating tumor cell resistance to growth factor inhibition and demonstrate that targeting the αvβ3/RalB/NfkB/Src signaling pathway will circumvent growth factor resistance of a wide range of cancers.

### Pharmaceutical Compositions

In alternative embodiments, the invention provides pharmaceutical compositions for practicing the methods of the invention, e.g., pharmaceutical compositions for overcoming or diminishing or preventing Growth Factor Inhibitor (GFI) resistance in a cell, or, a method for increasing the growth-inhibiting effectiveness of a Growth Factor inhibitor on a cell, or, a method for re-sensitizing a cell to a Growth Factor Inhibitor.

In alternative embodiments, compositions used to practice the methods of the invention are formulated with a pharmaceutically acceptable carrier. In alternative embodiments, the pharmaceutical compositions used to practice the methods of the invention can be administered parenterally, topically, orally or by local administration, such as by aerosol or transdermally. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration are well described in the scientific and patent literature, see, e.g., the latest edition of Remington's Pharmaceutical Sciences, Maack Publishing Co, Easton Pa. (“Remington's”).

Therapeutic agents used to practice the methods of the invention can be administered alone or as a component of a pharmaceutical formulation (composition). The compounds may be formulated for administration in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the compositions used to practice the methods of the invention include those suitable for oral/nasal, topical, parenteral, rectal, and/or intravaginal administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect.

Pharmaceutical formulations used to practice the methods of the invention can be prepared according to any method known to the art for the manufacture of pharmaceuticals. Such drugs can contain sweetening agents, flavoring agents, coloring agents and preserving agents. A formulation can be admixtured with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

Pharmaceutical formulations for oral administration can be formulated using pharmaceutically acceptable carriers well known in the art in appropriate and suitable dosages. Such carriers enable the pharmaceuticals to be formulated in unit dosage forms as tablets, geltabs, pills, powder, dragees, capsules, liquids, lozenges, gels, syrups, slurries, suspensions, etc., suitable for ingestion by the patient. Pharmaceutical preparations for oral use can be formulated as a solid excipient, optionally grinding a resulting mixture, and processing the mixture of granules, after adding suitable additional compounds, if desired, to obtain tablets or dragee cores. Suitable solid excipients are carbohydrate or protein fillers include, e.g., sugars, including lactose, sucrose, mannitol, or sorbitol; starch from corn, wheat, rice, potato, or other plants; cellulose such as methyl cellulose, hydroxypropylmethyl-cellulose, or sodium carboxy-methylcellulose; and gums including arabic and tragacanth; and proteins, e.g., gelatin and collagen. Disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations used to practice the methods of the invention can also be used orally using, e.g., push-fit capsules made of gelatin, as well as soft, sealed capsules made of gelatin and a coating such as glycerol or sorbitol. Push-fit capsules can contain active agents mixed with a filler or binders such as lactose or starches, lubricants such as talc or magnesium stearate, and, optionally, stabilizers. In soft capsules, the active agents can be dissolved or suspended in suitable liquids, such as fatty oils, liquid paraffin, or liquid polyethylene glycol with or without stabilizers.

Aqueous suspensions can contain an active agent (e.g., a composition used to practice the methods of the invention) in admixture with excipients suitable for the manufacture of aqueous suspensions. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Oil-based pharmaceuticals are particularly useful for administration hydrophobic active agents used to practice the methods of the invention. Oil-based suspensions can be formulated by suspending an active agent in a vegetable oil, such as arachis oil, olive oil, sesame oil or coconut oil, or in a mineral oil such as liquid paraffin; or a mixture of these. See e.g., U.S. Pat. No. 5,716,928 describing using essential oils or essential oil components for increasing bioavailability and reducing inter- and intra-individual variability of orally administered hydrophobic pharmaceutical compounds (see also U.S. Pat. No. 5,858,401). The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto (1997) J. Pharmacol. Exp. Ther. 281:93-102. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

In practicing this invention, the pharmaceutical compounds can also be administered by in intranasal, intraocular and intravaginal routes including suppositories, insufflation, powders and aerosol formulations (for examples of steroid inhalants, see Rohatagi (1995) J. Clin. Pharmacol. 35:1187-1193; Tjwa (1995) Ann. Allergy Asthma Immunol. 75:107-111). Suppositories formulations can be prepared by mixing the drug with a suitable non-irritating excipient which is solid at ordinary temperatures but liquid at body temperatures and will therefore melt in the body to release the drug. Such materials are cocoa butter and polyethylene glycols.

In practicing this invention, the pharmaceutical compounds can be delivered by transdermally, by a topical route, formulated as applicator sticks, solutions, suspensions, emulsions, gels, creams, ointments, pastes, jellies, paints, powders, and aerosols.

In practicing this invention, the pharmaceutical compounds can also be delivered as microspheres for slow release in the body. For example, microspheres can be administered via intradermal injection of drug which slowly release subcutaneously; see Rao (1995) J. Biomater Sci. Polym. Ed. 7:623-645; as biodegradable and injectable gel formulations, see, e.g., Gao (1995) Pharm. Res. 12:857-863 (1995); or, as microspheres for oral administration, see, e.g., Eyles (1997) J. Pharm. Pharmacol. 49:669-674.

In practicing this invention, the pharmaceutical compounds can be parenterally administered, such as by intravenous (IV) administration or administration into a body cavity or lumen of an organ. These formulations can comprise a solution of active agent dissolved in a pharmaceutically acceptable carrier. Acceptable vehicles and solvents that can be employed are water and Ringer's solution, an isotonic sodium chloride. In addition, sterile fixed oils can be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally-acceptable diluent or solvent, such as a solution of 1,3-butanediol. The administration can be by bolus or continuous infusion (e.g., substantially uninterrupted introduction into a blood vessel for a specified period of time).

The pharmaceutical compounds and formulations used to practice the methods of the invention can be lyophilized. The invention provides a stable lyophilized formulation comprising a composition of the invention, which can be made by lyophilizing a solution comprising a pharmaceutical of the invention and a bulking agent, e.g., mannitol, trehalose, raffinose, and sucrose or mixtures thereof. A process for preparing a stable lyophilized formulation can include lyophilizing a solution about 2.5 mg/mL protein, about 15 mg/mL sucrose, about 19 mg/mL NaCl, and a sodium citrate buffer having a pH greater than 5.5 but less than 6.5. See, e.g., U.S. patent app. no. 20040028670.

The compositions and formulations used to practice the methods of the invention can be delivered by the use of liposomes (see also discussion, below). By using liposomes, particularly where the liposome surface carries ligands specific for target cells, or are otherwise preferentially directed to a specific organ, one can focus the delivery of the active agent into target cells in vivo. See, e.g., U.S. Pat. Nos. 6,063,400; 6,007,839; Al-Muhammed (1996) J. Microencapsul. 13:293-306; Chonn (1995) Curr. Opin. Biotechnol. 6:698-708; Ostro (1989) Am. J. Hosp. Pharm. 46:1576-1587.

The formulations used to practice the methods of the invention can be administered for prophylactic and/or therapeutic treatments. In therapeutic applications, compositions are administered to a subject already suffering from a condition, infection or disease in an amount sufficient to cure, alleviate or partially arrest the clinical manifestations of the condition, infection or disease and its complications (a “therapeutically effective amount”). For example, in alternative embodiments, pharmaceutical compositions of the invention are administered in an amount sufficient to treat, prevent and/or ameliorate normal, dysfunction (e.g., abnormally proliferating) cell, e.g., cancer cell, or blood vessel cell, including endothelial and/or capillary cell growth; including neovasculature related to (within, providing a blood supply to) hyperplastic tissue, a granuloma or a tumor. The amount of pharmaceutical composition adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods of the invention are correct and appropriate.

Single or multiple administrations of formulations can be given depending on the dosage and frequency as required and tolerated by the patient. The formulations should provide a sufficient quantity of active agent to effectively treat, prevent or ameliorate a conditions, diseases or symptoms as described herein. For example, an exemplary pharmaceutical formulation for oral administration of compositions used to practice the methods of the invention can be in a daily amount of between about 0.1 to 0.5 to about 20, 50, 100 or 1000 or more ug per kilogram of body weight per day. In an alternative embodiment, dosages are from about 1 mg to about 4 mg per kg of body weight per patient per day are used. Lower dosages can be used, in contrast to administration orally, into the blood stream, into a body cavity or into a lumen of an organ. Substantially higher dosages can be used in topical or oral administration or administering by powders, spray or inhalation. Actual methods for preparing parenterally or non-parenterally administrable formulations will be known or apparent to those skilled in the art and are described in more detail in such publications as Remington's, supra.

The methods of the invention can further comprise co-administration with other drugs or pharmaceuticals, e.g., compositions for treating cancer, septic shock, infection, fever, pain and related symptoms or conditions. For example, the methods and/or compositions and formulations of the invention can be co-formulated with and/or co-administered with antibiotics (e.g., antibacterial or bacteriostatic peptides or proteins), particularly those effective against gram negative bacteria, fluids, cytokines, immunoregulatory agents, anti-inflammatory agents, complement activating agents, such as peptides or proteins comprising collagen-like domains or fibrinogen-like domains (e.g., a ficolin), carbohydrate-binding domains, and the like and combinations thereof.

### Nanoparticles and Liposomes

The invention also provides nanoparticles and liposomal membranes comprising compounds used to practice the methods of the invention. In alternative embodiments, the invention provides nanoparticles and liposomal membranes targeting diseased and/or tumor (cancer) stem cells and dysfunctional stem cells, and angiogenic cells.

In alternative embodiments, the invention provides nanoparticles and liposomal membranes comprising (in addition to comprising compounds used to practice the methods of the invention) molecules, e.g., peptides or antibodies, that selectively target abnormally growing, diseased, infected, dysfunctional and/or cancer (tumor) cell receptors. In alternative embodiments, the invention provides nanoparticles and liposomal membranes using IL-11 receptor and/or the GRP78 receptor to targeted receptors on cells, e.g., on tumor cells, e.g., on prostate or ovarian cancer cells. See, e.g., U.S. patent application publication no. 20060239968.

In one aspect, the compositions used to practice the methods of the invention are specifically targeted for inhibiting, ameliorating and/or preventing endothelial cell migration and for inhibiting angiogenesis, e.g., tumor-associated or disease- or infection-associated neovasculature.

The invention also provides nanocells to allow the sequential delivery of two different therapeutic agents with different modes of action or different pharmacokinetics, at least one of which comprises a composition used to practice the methods of the invention. A nanocell is formed by encapsulating a nanocore with a first agent inside a lipid vesicle containing a second agent; see, e.g., Sengupta, et al., U.S. Pat. Pub. No. 20050266067. The agent in the outer lipid compartment is released first and may exert its effect before the agent in the nanocore is released. The nanocell delivery system may be formulated in any pharmaceutical composition for delivery to patients suffering from a diseases or condition as described herein, e.g., such as a retinal age-related macular degeneration, a diabetic retinopathy, a cancer or carcinoma, a glioblastoma, a neuroma, a neuroblastoma, a colon carcinoma, a hemangioma, an infection and/or a condition with at least one inflammatory component, and/or any infectious or inflammatory disease, such as a rheumatoid arthritis, a psoriasis, a fibrosis, leprosy, multiple sclerosis, inflammatory bowel disease, or ulcerative colitis or Crohn's disease.

In treating cancer, a traditional antineoplastic agent is contained in the outer lipid vesicle of the nanocell, and an antiangiogenic agent of this invention is loaded into the nanocore. This arrangement allows the antineoplastic agent to be released first and delivered to the tumor before the tumor's blood supply is cut off by the composition of this invention.

The invention also provides multilayered liposomes comprising compounds used to practice this invention, e.g., for transdermal absorption, e.g., as described in Park, et al., U.S. Pat. Pub. No. 20070082042. The multilayered liposomes can be prepared using a mixture of oil-phase components comprising squalane, sterols, ceramides, neutral lipids or oils, fatty acids and lecithins, to about 200 to 5000 nm in particle size, to entrap a composition of this invention.

A multilayered liposome used to practice the invention may further include an antiseptic, an antioxidant, a stabilizer, a thickener, and the like to improve stability. Synthetic and natural antiseptics can be used, e.g., in an amount of 0.01% to 20%. Antioxidants can be used, e.g., BHT, erysorbate, tocopherol, astaxanthin, vegetable flavonoid, and derivatives thereof, or a plant-derived antioxidizing substance. A stabilizer can be used to stabilize liposome structure, e.g., polyols and sugars. Exemplary polyols include butylene glycol, polyethylene glycol, propylene glycol, dipropylene glycol and ethyl carbitol; examples of sugars are trehalose, sucrose, mannitol, sorbitol and chitosan, or a monosaccharides or an oligosaccharides, or a high molecular weight starch. A thickener can be used for improving the dispersion stability of constructed liposomes in water, e.g., a natural thickener or an acrylamide, or a synthetic polymeric thickener. Exemplary thickeners include natural polymers, such as acacia gum, xanthan gum, gellan gum, locust bean gum and starch, cellulose derivatives, such as hydroxy ethylcellulose, hydroxypropyl cellulose and carboxymethyl cellulose, synthetic polymers, such as polyacrylic acid, poly-acrylamide or polyvinylpyrollidone and polyvinylalcohol, and copolymers thereof or cross-linked materials.

Liposomes can be made using any method, e.g., as described in Park, et al., U.S. Pat. Pub. No. 20070042031, including method of producing a liposome by encapsulating a therapeutic product comprising providing an aqueous solution in a first reservoir; providing an organic lipid solution in a second reservoir, wherein one of the aqueous solution and the organic lipid solution includes a therapeutic product; mixing the aqueous solution with said organic lipid solution in a first mixing region to produce a liposome solution, wherein the organic lipid solution mixes with said aqueous solution so as to substantially instantaneously produce a liposome encapsulating the therapeutic product; and immediately thereafter mixing the liposome solution with a buffer solution to produce a diluted liposome solution.

The invention also provides nanoparticles comprising compounds used to practice this invention to deliver a composition of the invention as a drug-containing nanoparticles (e.g., a secondary nanoparticle), as described, e.g., in U.S. Pat. Pub. No. 20070077286. In one embodiment, the invention provides nanoparticles comprising a fat-soluble drug of this invention or a fat-solubilized water-soluble drug to act with a bivalent or trivalent metal salt.

Liposomes

The compositions and formulations used to practice the invention can be delivered by the use of liposomes. By using liposomes, particularly where the liposome surface carries ligands specific for target cells, or are otherwise preferentially directed to a specific organ, one can focus the delivery of the active agent into target cells in vivo. See, e.g., U.S. Pat. Nos. 6,063,400; 6,007,839; Al-Muhammed (1996) J. Microencapsul. 13:293-306; Chonn (1995) Curr. Opin. Biotechnol. 6:698-708; Ostro (1989) Am. J. Hosp. Pharm. 46:1576-1587. For example, in one embodiment, compositions and formulations used to practice the invention are delivered by the use of liposomes having rigid lipids having head groups and hydrophobic tails, e.g., as using a polyethyleneglycol-linked lipid having a side chain matching at least a portion the lipid, as described e.g., in US Pat App Pub No. 20080089928. In another embodiment, compositions and formulations used to practice the invention are delivered by the use of amphoteric liposomes comprising a mixture of lipids, e.g., a mixture comprising a cationic amphiphile, an anionic amphiphile and/or neutral amphiphiles, as described e.g., in US Pat App Pub No. 20080088046, or 20080031937. In another embodiment, compositions and formulations used to practice the invention are delivered by the use of liposomes comprising a polyalkylene glycol moiety bonded through a thioether group and an antibody also bonded through a thioether group to the liposome, as described e.g., in US Pat App Pub No. 20080014255. In another embodiment, compositions and formulations used to practice the invention are delivered by the use of liposomes comprising glycerides, glycerophospholipides, glycerophosphinolipids, glycerophosphonolipids, sulfolipids, sphingolipids, phospholipids, isoprenolides, steroids, stearines, sterols and/or carbohydrate containing lipids, as described e.g., in US Pat App Pub No. 20070148220.

### Antibodies as Pharmaceutical Compositions

In alternative embodiments, the invention provides compositions and methods for inhibiting or depleting an integrin αᵥβ₃ (anb3), or inhibiting an integrin aᵥβ₃(anb3) protein activity, or inhibiting the formation or activity of an integrin anb3/RalB signaling complex, or inhibiting the formation or signaling activity of an integrin αᵥβ₃ (anb3)/RalB/NFkB signaling axis; or inhibiting or depleting a RalB protein or an inhibitor of RalB protein activation; or inhibiting or depleting a Src or TBK1 protein or an inhibitor of Src or TBK1 protein activation. In alternative embodiments, this is achieved by administration of inhibitory antibodies. For example, in alternative embodiments, the invention uses isolated, synthetic or recombinant antibodies that specifically bind to and inhibit an integrin αᵥβ₃ (anb3), or any protein of an integrin aᵥβ₃ (anb3)/RalB/NFkB signaling axis, a RalB protein, a Src or TBK1 protein, or an NFkB protein.

In alternative aspects, an antibody for practicing the invention can comprise a peptide or polypeptide derived from, modeled after or substantially encoded by an immunoglobulin gene or immunoglobulin genes, or fragments thereof, capable of specifically binding an antigen or epitope, see, e.g. Fundamental Immunology, Third Edition, W.E. Paul, ed., Raven Press, N.Y. (1993); Wilson (1994) J. Immunol. Methods 175:267-273; Yarmush (1992) J. Biochem. Biophys. Methods 25:85-97. In alternative aspects, an antibody for practicing the invention includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody.”

In alternative embodiments, the invention uses “humanized” antibodies, including forms of non-human (e.g., murine) antibodies that are chimeric antibodies comprising minimal sequence (e.g., the antigen binding fragment) derived from non-human immunoglobulin. In alternative embodiments, humanized antibodies are human immunoglobulins in which residues from a hypervariable region (HVR) of a recipient (e.g., a human antibody sequence) are replaced by residues from a hypervariable region (HVR) of a non-human species (donor antibody) such as mouse, rat, rabbit or nonhuman primate having the desired specificity, affinity, and capacity. In alternative embodiments, framework region (FR) residues of the human immunoglobulin are replaced by corresponding non-human residues to improve antigen binding affinity.

In alternative embodiments, humanized antibodies may comprise residues that are not found in the recipient antibody or the donor antibody. These modifications may be made to improve antibody affinity or functional activity. In alternative embodiments, the humanized antibody can comprise substantially all of at least one, and typically two, variable domains, in which all or substantially all of the hypervariable regions correspond to those of a non-human immunoglobulin and all or substantially all of Ab framework regions are those of a human immunoglobulin sequence.

In alternative embodiments, a humanized antibody used to practice this invention can comprise at least a portion of an immunoglobulin constant region (Fc), typically that of or derived from a human immunoglobulin.

However, in alternative embodiments, completely human antibodies also can be used to practice this invention, including human antibodies comprising amino acid sequence which corresponds to that of an antibody produced by a human. This definition of a human antibody specifically excludes a humanized antibody comprising non-human antigen binding residues.

In alternative embodiments, antibodies used to practice this invention comprise “affinity matured” antibodies, e.g., antibodies comprising with one or more alterations in one or more hypervariable regions which result in an improvement in the affinity of the antibody for antigen; e.g., NFkB, an integrin αᵥβ₃ (anb3), or any protein of an integrin αᵥβ₃ (anb3)/RalB/NFkB signaling axis, a RalB protein, a Src or TBK1 protein, compared to a parent antibody which does not possess those alteration(s). In alternative embodiments, antibodies used to practice this invention are matured antibodies having nanomolar or even picomolar affinities for the target antigen, e.g., NFkB, an integrin αᵥβ₃ (anb3), or any protein of an integrin αᵥβ₃ (anb3)/RalB/NFkB signaling axis, a RalB protein, a Src or TBK1 protein. Affinity matured antibodies can be produced by procedures known in the art.

Antisense, siRNAs and microRNAs as Pharmaceutical Compositions

In alternative embodiments, the invention provides compositions and methods for inhibiting or depleting an integrin αᵥβ₃ (anb3), or inhibiting an integrin αᵥβ₃ (anb3) protein activity, or inhibiting the formation or activity of an integrin anb3/RalB signaling complex, or inhibiting the formation or signaling activity of an integrin αᵥβ₃ (anb3)/RalB/NFkB signaling axis; or inhibiting or depleting a RalB protein or an inhibitor of RalB protein activation; or inhibiting or depletihg a Src or TBK1 protein or an inhibitor of Src or TBK1 protein activation. In alternative embodiments, this is achieved by administration of inhibitory nucleic acids, e.g., siRNA, antisense nucleic acids, and/or inhibitory microRNAs.

In alternative embodiments, compositions used to practice the invention are formulated with a pharmaceutically acceptable carrier. In alternative embodiments, the pharmaceutical compositions used to practice the invention can be administered parenterally, topically, orally or by local administration, such as by aerosol or transdermally. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration are well described in the scientific and patent literature, see, e.g., the latest edition of Remington's Pharmaceutical Sciences, Maack Publishing Co, Easton Pa. (“Remington's”).

While the invention is not limited by any particular mechanism of action: microRNAs (miRNAs) are short (20-24 nt) non-coding RNAs that are involved in post-transcriptional regulation of gene expression in multicellular organisms by affecting both the stability and translation of mRNAs. miRNAs are transcribed by RNA polymerase II as part of capped and polyadenylated primary transcripts (pri-miRNAs) that can be either protein-coding or non-coding. The primary transcript is cleaved by the Drosha ribonuclease III enzyme to produce an approximately 70-nt stem-loop precursor miRNA (pre-miRNA), which is further cleaved by the cytoplasmic Dicer ribonuclease to generate the mature miRNA and antisense miRNA star (miRNA*) products. The mature miRNA is incorporated into a RNA-induced silencing complex (RISC), which recognizes target mRNAs through imperfect base pairing with the miRNA and most commonly results in translational inhibition or destabilization of the target mRNA.

In alternative embodiments pharmaceutical compositions used to practice the invention are administered in the form of a dosage unit, e.g., a tablet, capsule, bolus, spray. In alternative embodiments, pharmaceutical compositions comprise a compound, e.g., an antisense nucleic acid, e.g., an siRNA or a microRNA, in a dose: e.g., 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 270 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, or 800 mg or more.

In alternative embodiments, an siRNA or a microRNA used to practice the invention is administered as a pharmaceutical agent, e.g., a sterile formulation, e.g., a lyophilized siRNA or microRNA that is reconstituted with a suitable diluent, e.g., sterile water for injection or sterile saline for injection. In alternative embodiments the reconstituted product is administered as a subcutaneous injection or as an intravenous infusion after dilution into saline. In alternative embodiments the lyophilized drug product comprises siRNA or microRNA prepared in water for injection, or in saline for injection, adjusted to pH 7.0-9.0 with acid or base during preparation, and then lyophilized. In alternative embodiments a lyophilized siRNA or microRNA of the invention is between about 25 to 800 or more mg, or about 25, 50, 75, 100, 125, 150, 175, 200, 225, 250, 275, 300, 325, 350, 375, 425, 450, 475, 500, 525, 550, 575, 600, 625, 650, 675, 700, 725, 750, 775, and 800 mg of a siRNA or microRNA of the invention. The lyophilized siRNA or microRNA of the invention can be packaged in a 2 mL Type 1, clear glass vial (e.g., ammonium sulfate-treated), e.g., stoppered with a bromobutyl rubber closure and sealed with an aluminum overseal.

In alternative embodiments, the invention provides compositions and methods comprising in vivo delivery of antisense nucleic acids, e.g., siRNA or microRNAs. In practicing the invention, the antisense nucleic acids, siRNAs, or microRNAs can be modified, e.g., in alternative embodiments, at least one nucleotide of antisense nucleic acid, e.g., siRNA or microRNA, construct is modified, e.g., to improve its resistance to nucleases, serum stability, target specificity, blood system circulation, tissue distribution, tissue penetration, cellular uptake, potency, and/or cell-permeability of the polynucleotide. In alternative embodiments, the antisense nucleic acid, siRNA or microRNA construct is unmodified. In other embodiments, at least one nucleotide in the antisense nucleic acid, siRNA or microRNA construct is modified.

In alternative embodiments, guide strand modifications are made to increase nuclease stability, and/or lower interferon induction, without significantly decreasing antisense nucleic acid, siRNA or microRNA activity (or no decrease in antisense nucleic acid, siRNA or microRNA activity at all). In certain embodiments, the modified antisense nucleic acid, siRNA or microRNA constructs have improved stability in serum and/or cerebral spinal fluid compared to an unmodified structure having the same sequence.

In alternative embodiments, a modification includes a 2′-H or 2′-modified ribose sugar at the second nucleotide from the 5′-end of the guide sequence. In alternative embodiments, the guide strand (e.g., at least one of the two single-stranded polynucleotides) comprises a 2′-O-alkyl or 2′-halo group, such as a 2′-O-methyl modified nucleotide, at the second nucleotide on the 5′-end of the guide strand, or, no other modified nucleotides. In alternative embodiments, polynucleotide constructs having such modification may have enhanced target specificity or reduced off-target silencing compared to a similar construct without the 2′-O-methyl modification at the position.

In alternative embodiments, a second nucleotide is a second nucleotide from the 5′-end of the single-stranded polynucleotide. In alternative embodiments, a “2′-modified ribose sugar” comprises ribose sugars that do not have a 2′-OH group. In alternative embodiments, a “2′-modified ribose sugar” does not include 2′-deoxyribose (found in unmodified canonical DNA nucleotides), although one or more DNA nucleotides may be included in the subject constructs (e.g., a single deoxyribonucleotide, or more than one deoxyribonucleotide in a stretch or scattered in several parts of the subject constructs). For example, the 2′-modified ribose sugar may be 2′-O-alkyl nucleotides, 2′-deoxy-2′-fluoro nucleotides, 2′-deoxy nucleotides, or combination thereof.

In alternative embodiments, an antisense nucleic acid, siRNA or microRNA construct used to practice the invention comprises one or more 5′-end modifications, e.g., as described above, and can exhibit a significantly (e.g., at least about 25%, 30%, 35%, 40%, 45%, 50%, 55%, 60%, 65%, 70%, 75%, 80%, 85%, 90% or more) less “off-target” gene silencing when compared to similar constructs without the specified 5′-end modification, thus greatly improving the overall specificity of the antisense nucleic acid, siRNA or microRNA construct of the invention.

In alternative embodiments, an antisense nucleic acid, siRNA or microRNA construct to practice the invention comprises a guide strand modification that further increase stability to nucleases, and/or lowers interferon induction, without significantly decreasing activity (or no decrease in microRNA activity at all). In alternative embodiments, the 5′-stem sequence comprises a 2′-modified ribose sugar, such as 2′-O-methyl modified nucleotide, at the second nucleotide on the 5′-end of the polynucleotide, or, no other modified nucleotides. In alternative embodiments the hairpin structure having such modification has enhanced target specificity or reduced off-target silencing compared to a similar construct without the 2′-O-methyl modification at same position.

In alternative embodiments, the 2′-modified nucleotides are some or all of the pyrimidine nucleotides (e.g., C/U). Examples of 2′-O-alkyl nucleotides include a 2′-O-methyl nucleotide, or a 2′-O-allyl nucleotide. In alternative embodiments, the modification comprises a 2′-O-methyl modification at alternative nucleotides, starting from either the first or the second nucleotide from the 5′-end. In alternative embodiments, the modification comprises a 2′-O-methyl modification of one or more randomly selected pyrimidine nucleotides (C or U). In alternative embodiments, the modification comprises a 2′-O-methyl modification of one or more nucleotides within the loop.

In alternative embodiments, the modified nucleotides are modified on the sugar moiety, the base, and/or the phosphodiester linkage. In alternative embodiments the modification comprise a phosphate analog, or a phosphorothioate linkage; and the phosphorothioate linkage can be limited to one or more nucleotides within the loop, a 5′-overhang, and/or a 3′-overhang.

In alternative embodiments, the phosphorothioate linkage may be limited to one or more nucleotides within the loop, and 1, 2, 3, 4, 5, or 6 more nucleotide(s) of the guide sequence within the double-stranded stem region just 5′ to the loop. In alternative embodiments, the total number of nucleotides having the phosphorothioate linkage may be about 12-14. In alternative embodiments, all nucleotides having the phosphorothioate linkage are not contiguous. In alternative embodiments, the modification comprises a 2′-O-methyl modification, or, no more than 4 consecutive nucleotides are modified. In alternative embodiments, all nucleotides in the 3′-end stem region are modified. In alternative embodiments, all nucleotides 3′ to the loop are modified.

In alternative embodiments, the 5′- or 3′-stem sequence comprises one or more universal base-pairing nucleotides. In alternative embodiments universal base-pairing nucleotides include extendable nucleotides that can be incorporated into a polynucleotide strand (either by chemical synthesis or by a polymerase), and pair with more than one pairing type of specific canonical nucleotide. In alternative embodiments, the universal nucleotides pair with any specific nucleotide. In alternative embodiments, the universal nucleotides pair with four pairings types of specific nucleotides or analogs thereof. In alternative embodiments, the universal nucleotides pair with three pairings types of specific nucleotides or analogs thereof. In alternative embodiments, the universal nucleotides pair with two pairings types of specific nucleotides or analogs thereof.

In alternative embodiments, an antisense nucleic acid, siRNA or microRNA used to practice the invention comprises a modified nucleoside, e.g., a sugar-modified nucleoside. In alternative embodiments, the sugar-modified nucleosides can further comprise a natural or modified heterocyclic base moiety and/or a natural or modified internucleoside linkage; or can comprise modifications independent from the sugar modification. In alternative embodiments, a sugar modified nucleoside is a 2′-modified nucleoside, wherein the sugar ring is modified at the 2′ carbon from natural ribose or 2′-deoxy-ribose.

In alternative embodiments, a 2′-modified nucleoside has a bicyclic sugar moiety. In certain such embodiments, the bicyclic sugar moiety is a D sugar in the alpha configuration. In certain such embodiments, the bicyclic sugar moiety is a D sugar in the beta configuration. In certain such embodiments, the bicyclic sugar moiety is an L sugar in the alpha configuration. In alternative embodiments, the bicyclic sugar moiety is an L sugar in the beta configuration.

In alternative embodiments, the bicyclic sugar moiety comprises a bridge group between the 2′ and the 4′-carbon atoms. In alternative embodiments, the bridge group comprises from 1 to 8 linked biradical groups. In alternative embodiments, the bicyclic sugar moiety comprises from 1 to 4 linked biradical groups. In alternative embodiments, the bicyclic sugar moiety comprises 2 or 3 linked biradical groups.

In alternative embodiments, the bicyclic sugar moiety comprises 2 linked biradical groups. In alternative embodiments, a linked biradical group is selected from —O—, —S—, —N(R1)-, —C(R1)(R2)-, —C(R1)=C(R1)-, —C(R1)=N—, —C(═NR1)-, —Si(R1)(R₂)—, —S(═O)₂—, —S(═O)—, —C(═O)— and —C(═S)—; where each R1 and R₂ is, independently, H, hydroxyl, C1 to C₁₂ alkyl, substituted C1-C12 alkyl, C₂-C12 alkenyl, substituted C₂-C₁₂ alkenyl, C₂-C₁₂ alkynyl, substituted C₂-C12 alkynyl, C₂-C20 aryl, substituted C₂-C20 aryl, a heterocycle radical, a substituted heterocycle radical, heteroaryl, substituted heteroaryl, C₂-C₇ alicyclic radical, substituted C₂-C₇ alicyclic radical, halogen, substituted oxy (—O—), amino, substituted amino, azido, carboxyl, substituted carboxyl, acyl, substituted acyl, CN, thiol, substituted thiol, sulfonyl (S(═O)₂—H), substituted sulfonyl, sulfoxyl (S(═O)—H) or substituted sulfoxyl; and each substituent group is, independently, halogen, C1-C₁₂ alkyl, substituted C1-C₁₂ alkyl, C₂-C₁₂ alkenyl, substituted C₂-C₁₂ alkenyl, C₂-C₁₂ alkynyl, substituted C₂-C₁₂ alkynyl, amino, substituted amino, acyl, substituted acyl, C1-C₁₂ aminoalkyl, C1-C₁₂ aminoalkoxy, substituted C1-C₁₂ aminoalkyl, substituted C1-C₁₂ aminoalkoxy or a protecting group.

In alternative embodiments, the bicyclic sugar moiety is bridged between the 2′ and 4′ carbon atoms with a biradical group selected from —O—(CH₂)x-, —O—CH₂—, —O—CH₂CH₂—, —O—CH(alkyl)-, —NH—(CH2)P—, —N(alkyl)-(CH₂)x-, —O—CH(alkyl)-, —(CH(alkyl))—(CH2)x-, —NH—O—(CH2)x-, —N(alkyl)-O—(CH₂)x-, or —O—N(alkyl)-(CH₂)x-, wherein x is 1, 2, 3, 4 or 5 and each alkyl group can be further substituted. In certain embodiments, x is 1, 2 or 3.

In alternative embodiments, a 2′-modified nucleoside comprises a 2′-substituent group selected from halo, allyl, amino, azido, SH, CN, OCN, CF₃, OCF₃, O—, S—, or N(Rm)-alkyl; O—, S—, or N(Rm)-alkenyl; O—, S— or N(Rm)-alkynyl; O-alkylenyl-O-alkyl, alkynyl, alkaryl, aralkyl, O-alkaryl, O-aralkyl, O(CH₂)₂SCH₃, O—(CH₂)₂—O—N(Rm)(Rn) or O—CH2-C(═O)—N(Rm)(Rn), where each Rm and Rn is, independently, H, an amino protecting group or substituted or unsubstituted C1-C10 alkyl. These 2′-substituent groups can be further substituted with one or more substituent groups independently selected from hydroxyl, amino, alkoxy, carboxy, benzyl, phenyl, nitro (NO.sub.2), thiol, thioalkoxy (S-alkyl), halogen, alkyl, aryl, alkenyl and alkynyl.

In alternative embodiments, a 2′-modified nucleoside comprises a 2′-substituent group selected from F, O—CH₃, and OCH₂CH2OCH₃.

In alternative embodiments, a sugar-modified nucleoside is a 4′-thio modified nucleoside. In alternative embodiments, a sugar-modified nucleoside is a 4′-thio-2′-modified nucleoside. In alternative embodiments a 4′-thio modified nucleoside has a .beta.-D-ribonucleoside where the 4′-0 replaced with 4′-S. A 4′-thio-2′-modified nucleoside is a 4′-thio modified nucleoside having the 2′-OH replaced with a 2′-substituent group. In alternative embodiments 2′-substituent groups include 2′-OCH3, 2′-O—(CH2).sub.2-OCH3, and 2′-F.

In alternative embodiments, a modified oligonucleotide of the present invention comprises one or more internucleoside modifications. In alternative embodiments, each internucleoside linkage of a modified oligonucleotide is a modified internucleoside linkage. In alternative embodiments, a modified internucleoside linkage comprises a phosphorus atom.

In alternative embodiments, a modified antisense nucleic acid, siRNA or microRNA comprises at least one phosphorothioate internucleoside linkage. In certain embodiments, each internucleoside linkage of a modified oligonucleotide is a phosphorothioate internucleoside linkage.

In alternative embodiments, a modified internucleoside linkage does not comprise a phosphorus atom. In alternative embodiments, an internucleoside linkage is formed by a short chain alkyl internucleoside linkage. In alternative embodiments, an internucleoside linkage is formed by a cycloalkyl internucleoside linkages. In alternative embodiments, an internucleoside linkage is formed by a mixed heteroatom and alkyl internucleoside linkage. In alternative embodiments, an internucleoside linkage is formed by a mixed heteroatom and cycloalkyl internucleoside linkages. In alternative embodiments, an internucleoside linkage is formed by one or more short chain heteroatomic internucleoside linkages. In alternative embodiments, an internucleoside linkage is formed by one or more heterocyclic internucleoside linkages. In alternative embodiments, an internucleoside linkage has an amide backbone, or an internucleoside linkage has mixed N, O, S and CH2 component parts.

In alternative embodiments, a modified oligonucleotide comprises one or more modified nucleobases. In certain embodiments, a modified oligonucleotide comprises one or more 5-methylcytosines, or each cytosine of a modified oligonucleotide comprises a 5-methylcytosine.

In alternative embodiments, a modified nucleobase comprises a 5-hydroxymethyl cytosine, 7-deazaguanine or 7-deazaadenine, or a modified nucleobase comprises a 7-deaza-adenine, 7-deazaguanosine, 2-aminopyridine or a 2-pyridone, or a modified nucleobase comprises a 5-substituted pyrimidines, 6-azapyrimidines and N-2, N-6 and O-6 substituted purines, or a 2 aminopropyladenine, 5-propynyluracil or a 5-propynylcytosine.

In alternative embodiments, a modified nucleobase comprises a polycyclic heterocycle, or a tricyclic heterocycle; or, a modified nucleobase comprises a phenoxazine derivative, or a phenoxazine further modified to form a nucleobase or G-clamp.

### Therapeutically Effective Amount and Doses

In alternative embodiment, compounds, compositions, pharmaceutical compositions and formulations used to practice the invention can be administered for prophylactic and/or therapeutic treatments; for example, the invention provides compositions and methods for overcoming or diminishing or preventing Growth Factor Inhibitor (GFI) resistance in a cell, or, a method for increasing the growth-inhibiting effectiveness of a Growth Factor inhibitor on a cell, or, a method for re-sensitizing a cell to a Growth Factor Inhibitor. In alternative embodiments, the invention provides compositions and methods for treating, preventing or ameliorating: a disease or condition associated with dysfunctional stem cells or cancer stem cells, a retinal age-related macular degeneration, a diabetic retinopathy, a cancer or carcinoma, a glioblastoma, a neuroma, a neuroblastoma, a colon carcinoma, a hemangioma, an infection and/or a condition with at least one inflammatory component, and/or any infectious or inflammatory disease, such as a rheumatoid arthritis, a psoriasis, a fibrosis, leprosy, multiple sclerosis, inflammatory bowel disease, or ulcerative colitis or Crohn's disease. In therapeutic applications, compositions are administered to a subject already suffering from a condition, infection or disease in an amount sufficient to cure, alleviate or partially arrest the clinical manifestations of the condition, infection or disease (e.g., disease or condition associated with dysfunctional stem cells or cancer stem cells) and its complications (a “therapeutically effective amount”). In the methods of the invention, a pharmaceutical composition is administered in an amount sufficient to treat (e.g., ameliorate) or prevent a disease or condition associated with dysfunctional stem cells or cancer stem cells. The amount of pharmaceutical composition adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

### Kits and Instructions

The invention provides kits comprising compositions for practicing the methods of the invention, including instructions for use thereof. In alternative embodiments, the invention provides kits, blister packages, lidded blisters or blister cards or packets, clamshells, trays or shrink wraps comprising a combination of compounds, wherein the combination of compounds comprises:

(1) at least one compound comprising or consisting of:
(i) an inhibitor or depleter of integrin αᵥβ₃ (anb3), or an inhibitor of integrin αᵥβ₃ (anb3) protein activity, or an inhibitor of the formation or activity of an integrin anb3/RalB signaling complex, or an inhibitor of the formation or signaling activity of an integrin αᵥβ₃ (anb3)/RalB/NFkB signaling axis,
wherein the inhibitor of integrin αᵥβ₃ protein activity is an allosteric inhibitor of integrin αᵥβ₃ protein activity;
(ii) an inhibitor or depleter of RalB protein or an inhibitor of RalB protein activation,
wherein the inhibitor of RalB protein activity is an allosteric inhibitor of RalB protein activity;
(iii) an inhibitor or depleter of Src or TBK1 protein or an inhibitor of Src or TBK1 protein activation,
wherein the inhibitor of Src or TBK1 protein activity is an allosteric inhibitor of Src or TBK1 protein activity;
(iv) an inhibitor or depleter of NFKB or IRF3 protein or an inhibitor of RalB protein activation,
wherein the inhibitor of NFKB or IRF3 protein activity is an allosteric inhibitor of NFKB or IRF3 protein activity; or
(v) any combination of (i) to (iv); and

(2) at least one Growth Factor Inhibitor.

In alternative embodiments, the kit further comprises instructions for practicing a method of the invention.

The invention will be further described with reference to the following examples; however, it is to be understood that the invention is not limited to such examples.

### EXAMPLES

### Example 1

### Methods of the Invention are Effective for Inhibiting and/or Promoting Cell Growth and Arresting Mitosis

The data presented herein demonstrates the effectiveness of the compositions and methods of the invention in sensitizing and re-sensitizing cancer cells, and cancer stem cells, to growth factor inhibitors, and validates this invention's therapeutic approach to overcome growth factor inhibitor, e.g., EGFR inhibitor, resistance for a wide range of cancers. The data presented herein demonstrates that genetic and pharmacological inhibition of RalB or NF-κB was able to re-sensitize αvβ3-expressing tumors to EGFR inhibitors.

Resistance to epidermal growth factor receptor (EGFR) inhibitors has emerged as a significant clinical problem in oncology owing to various resistance mechanisms^{1,2}. Since cancer stem cells have been associated with drug resistance³, we examined the expresion of stem/progenitor cell markers for breast, pancreas and colon tumor cells with acquired resistance to EGFR inhibitors. We found that CD61 (β3 integrin) was the one marker consistently upregulated on EGFR inhibitor resistant tumor cells. Moreover, integrin αvβ3 expression was markedly enhanced in murine orthotopic lung and pancreas tumors following their acquired resistance to systemically delivered EGFR inhibitors. In fact, αvβ3 was both necessary and sufficient to account for the tumor cell resistance to EGFR inhibitors and other growth factor receptor inhibitors but not cytotoxic drugs.

Mechanistically, in drug resistant tumors αvβ3 forms a complex with KRAS via the adaptor Galectin-3 resulting in recruitment of RalB and activation of its effector TBK1/NF-κB, revealing a previously undescribed integrin-mediated pathway. Accordingly, genetic or pharmacological inhibition of RalB or NF-κB was able to re-sensitize αvβ3-expressing tumors to EGFR inhibitors, demonstrating the effectiveness of the compositions and methods of the invention and validating this invention's therapeutic approach to overcome EGFR inhibitor resistance for a wide range of cancers.

Despite some level of clinical success achieved with EGFR Tyrosine Kinase inhibitors (TKIs), intrinsic and acquired cellular resistance mechanisms limit their efficacy^{1,2,4}. A number of resistance mechanisms have been identified, including KRAS and EGFR mutations, resulting in constitutive activation of the ERK pathway⁵⁻⁷. While KRAS-mediated ERK signaling is associated with resistance to EGFR inhibition, KRAS also induces PI3K and Ral activation leading to tumor cell survival and proliferation^{8,9}.

Nevertheless, it is clear that treatment of tumors with EGFR inhibitors appears to select for a cell population that remains insensitive to EGFR blockade^{1,2}. Prolonged administration of tumors with EGFR TKIs also selects for cells characterized by a distinct array of membrane proteins, including cancer stem/progenitor cell markers known to be associated with increased cell survival and metastasis¹⁰. While a number of EGFR-inhibitor resistance mechanisms have been defined, it is not clear whether a single unifying mechanism might drive the resistance of a broad range of cancers.

To investigate this, we exposed pancreatic (FG, Miapaca-2), breast (BT474, SKBR3 and MDAMB468) and colon (SW480) human tumor cell lines to increasing concentrations of erlotinib or lapatinib for three weeks, to select cell subpopulations that were at least 10-fold more resistant to these targeted therapies than their parental counterparts. Parent or resistant cells were then evaluated for a panel of stem/progenitor cell markers previously identified to be upregulated in the most aggressive metastatic tumor cells¹¹⁻¹³.

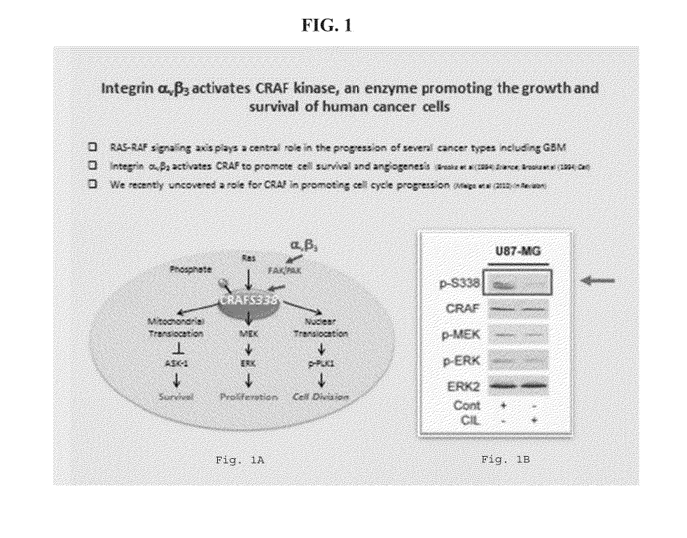

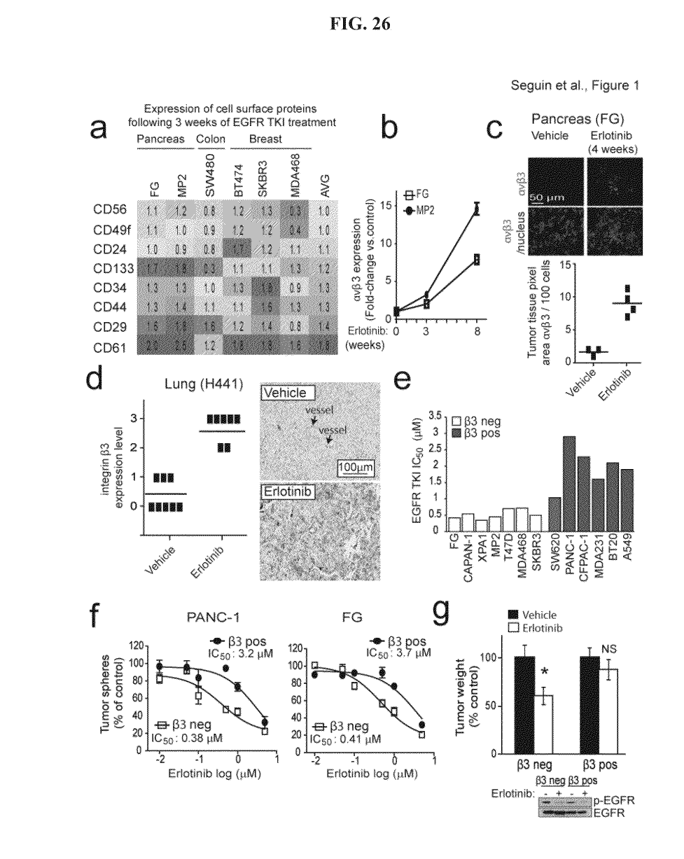

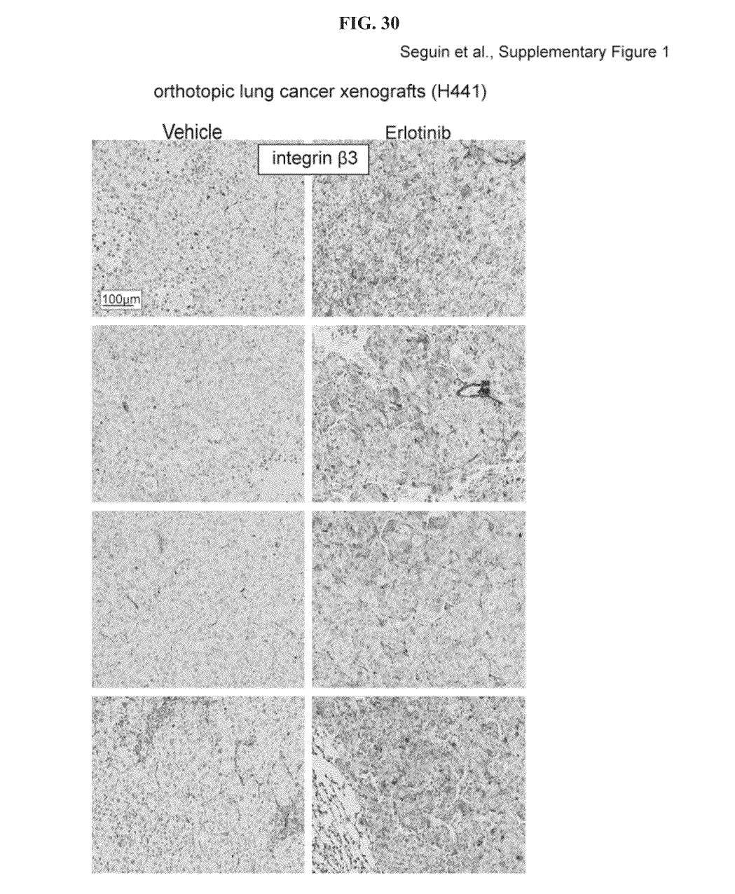

As expected, the expression of some of these markers was significantly increased in one or more of these resistant cell populations. Surprisingly, we observed that CD61 (integrin β3) was the one marker upregulated in all resistant cell lines tested, FIG. 26a. The longer cells were exposed to erlotinib the greater the expression level of αvβ3 was observed, FIG. 26b. These findings were extended in vivo as mice bearing orthotopic FG pancreatic tumors with minimal integrin αvβ3 evaluated following four weeks of erlotinib treatment showed a 10-fold increase in αvβ3 expression, FIG. 26c. Moreover, H441 human lung adenocarcinoma orthotopic tumors¹⁴ exposed to systemic erlotinib treatment in vivo for 7-8 weeks developed resistance and a qualitative increase in integrin αvβ3 expression compared with vehicle-treated tumors, see FIG. 26d and FIG. 30 (Supplementary FIG. 1). Thus, exposure of histologically distinct tumor cells in vitro or in vivo to EGFR inhibitors selects for a tumor cell population expressing high levels of αvβ3.

In addition to being expressed on a subpopulation of stem/progenitor cells during mammary development¹⁵, αvβ3 is a marker of the most malignant tumor cells in a wide range of cancers^{16,17}. To determine whether endogenous expression of integrin αvβ3 might predict tumor cell resistance to EGFR blockade, various breast, lung and pancreatic tumor cells were first screened for αvβ3 expression and then analyzed for their sensitivity to EGFR inhibitors (Supplementary Table 1).
Seguin et al., Supplementary Table 1

TABLE 1
KRAS mutation, integrin αvβ3 expression and
EGFR TKI sensitivity of cancer cell lines

| | | | integrin | |
|---|---|---|---|---|
| | | Mutated | αvβ3 | EGFR TKI |
| Cell line | Origin | KRAS | expression | sensitive |
| PANC-1 | pancreas | yes | [symbol] | [symbol] |
| FG | pancreas | yes | no | yes |
| Mapaca-2 (MP2) | pancreas | yes | no | yes |
| CAPAN-1 | pancreas | yes | no | yes |
| XPA-1 | pancreas | no | no | yes |
| CFPAC-1 | pancreas | yes | [symbol] | [symbol] |
| A549 | lung | yes | [symbol] | [symbol] |
| SKBR3 | breast | no | no | yes |
| MDAMB231 | breast | yes | [symbol] | [symbol] |
| MDAMB468 (MDA468) | breast | no | no | yes |
| BT474 | breast | no | no | yes |
| BT20 | breast | no | [symbol] | [symbol] |
| T47D | breast | yes | no | yes |
| SW480 | colon | yes | no | yes |

In all cases, β3 expressing tumor cells were intrinsically more resistant to EGFR blockade than β3-negative tumor cell lines (FIG. 26e). In fact, αvβ3 was required for resistance to EGFR inhibitors, since knockdown of αvβ3 in PANC-1 cells resulted in a 10-fold increase in tumor cell sensitivity to erlotinib (FIG. 260. Moreover, integrin αvβ3 was sufficient to induce erlotinib resistance since ectopic expression of αvβ3 in FG cells lacking this integrin dramatically increased erlotinib resistance both, in vitro and in orthotopic pancreatic tumors after systemic treatment in vivo (FIGS. 26f and g).

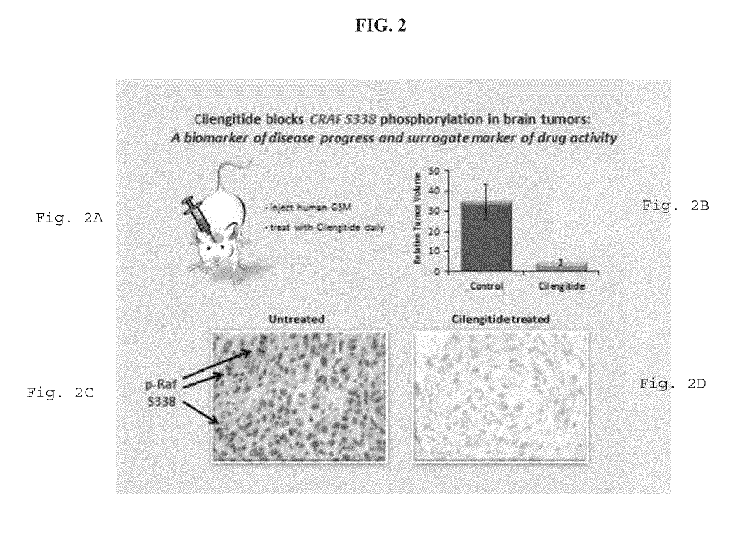

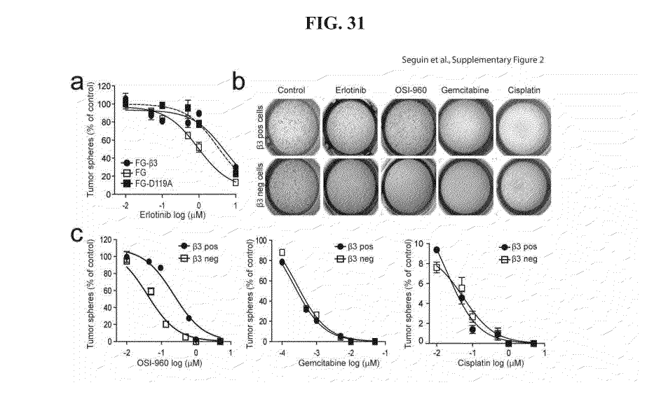

Integrin αvβ3 not only promotes adhesion-dependent signaling via activation of focal adhesion kinase FAK¹⁶ but it can also activate a FAK-independent signaling cascade in the absence of integrin ligation that is associated with increased survival and tumor metastasis¹⁷. To determine whether αvβ3 ligation was required for its causative role in erlotinib resistance, FG cells transfected with either WT β3 or a ligation deficient mutant of the integrin (D119A)¹⁷ were treated with erlotinib. The same degree of erlotinib resistance was observed in cells expressing either the ligation competent or incompetent form of integrin αvβ3, see FIG. 31a (Supplementary FIG. 2a) indicating that expression of αvβ3, even in the unligated state, was sufficient to induce tumor cell resistance to erlotinib.

Tumor cells with acquired resistance to one drug can often display resistance to a wide range of drugs^{18,19}. Therefore, we examined whether αvβ3 expression also promotes resistance to other growth factor inhibitors and/or cytotoxic agents. Interestingly, while αvβ3 expression accounted for EGFR inhibitor resistance, it also induced resistance to the IGFR inhibitor OSI-906, yet failed to protect cells from the antimetabolite agent gemcitabine and the chemotherapeutic agent cisplatin, see FIG. 31b and FIG. 31c (Supplementary FIGS. 2b and c). These results demonstrate that integrin αvβ3 accounts for tumor cell resistance to drugs that target growth factor receptor mediated pathways but does not promote for a more general resistant phenotype to all drugs, particularly those that induce cell cytotoxicity.

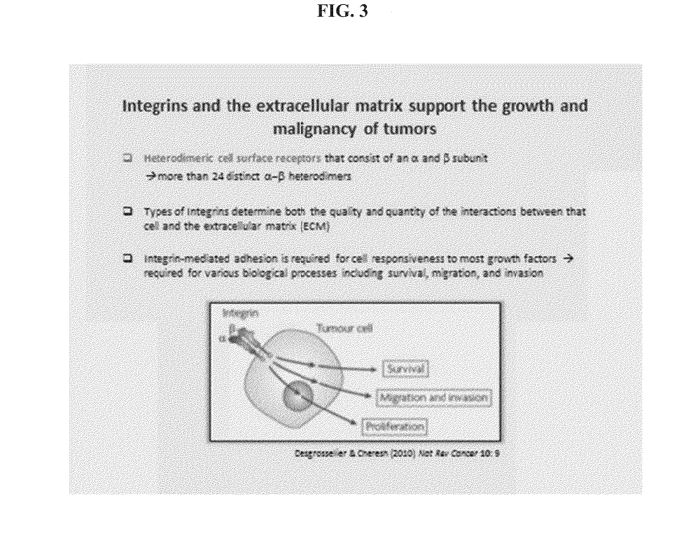

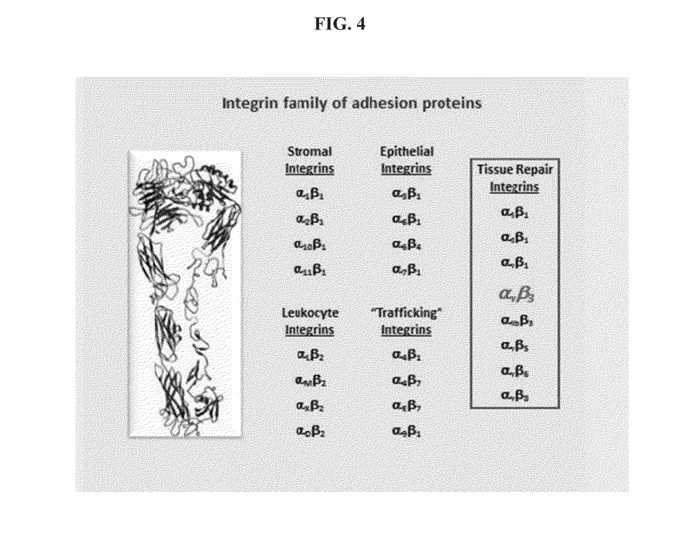

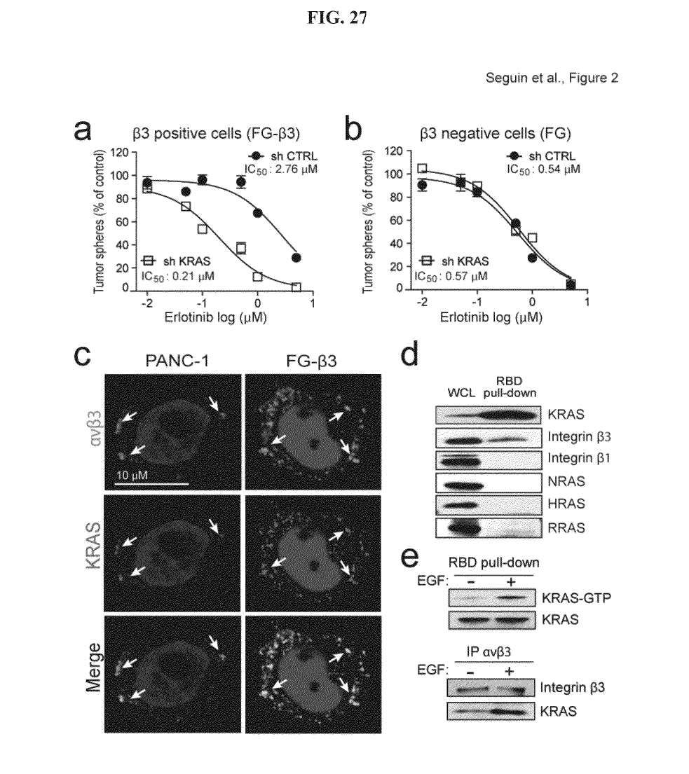

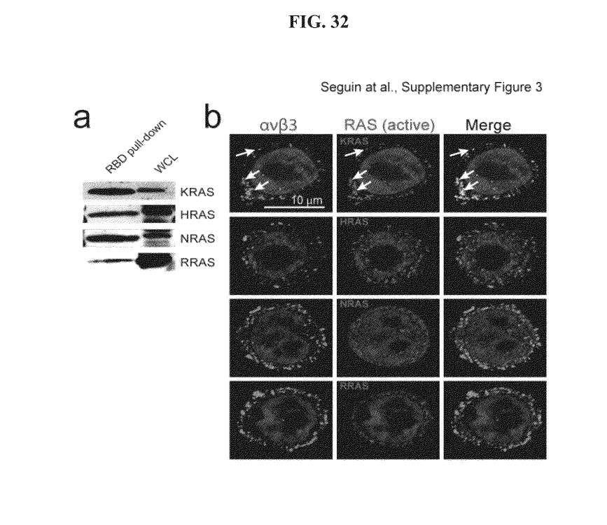

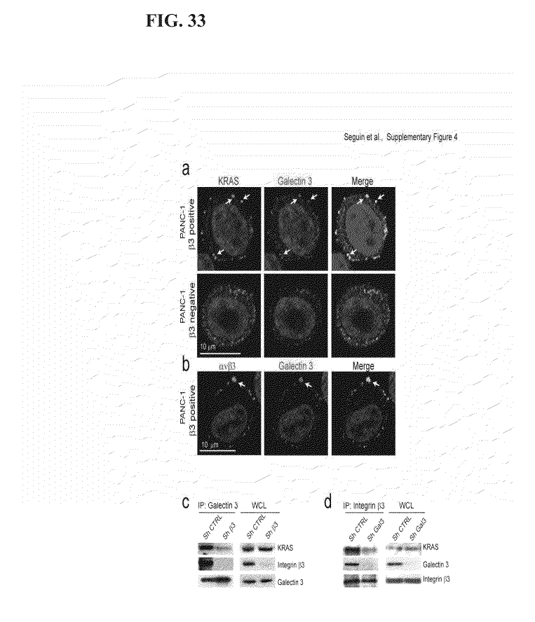

In some cases oncogenic KRAS has been associated with EGFR TKIs resistance²⁰, however, it remains unclear whether oncogenic KRAS is a prerequisite for EGFR resistance²¹. Thus, we examined the KRAS mutational status in various tumor cell lines and found that KRAS oncogenic status did not account for resistance to EGFR inhibitors (Supplementary Table 1). Nevertheless, knockdown of KRAS in αvβ3 expressing cells rendered them sensitive to erlotinib while KRAS knockdown in cells lacking αvβ3 had no such effect, see FIG. 31a and FIG. 31b, indicating that αvβ3 and KRAS function cooperatively to promote tumor cell resistance to erlotinib. Interestingly, even in non-adherent cells, αvβ3 colocalized with oncogenic KRAS in the plasma membrane (FIG. 27c) and could be co-precipitated in a complex with KRAS, see FIG. 31d. This interaction was specific for KRAS, as αvβ3 was not found to associate with N-, R- or H-RAS isoforms in these cells, see FIG. 31d and FIG. 32a and FIG. 32b (Supplementary FIGS. 3a and b). Furthermore, in BXPC3 human pancreatic tumor cells expressing wildtype KRAS, αvβ3 showed increased association with KRAS only after these cells were stimulated with EGF, see FIG. 31e. Previous studies have indicated that the KRAS interacting protein Galectin-3 can also couple to integrins^{22,23}. Therefore, we considered whether Galectin-3 might serve as an adaptor facilitating an interaction between αvβ3 and KRAS in epithelial tumor cells. In PANC-1 cells with endogenous β3 expression, αvβ3, KRAS, and Galectin-3 co-localized to membrane clusters, see FIG. 33a and FIG. 33b (Supplementary FIG. 4a-b). Furthermore, knockdown of either β3 or Galectin-3 prevented the localization of KRAS to these membrane clusters or their co-immunoprecipitation, see FIG. 33 (Supplementary FIG. 4).

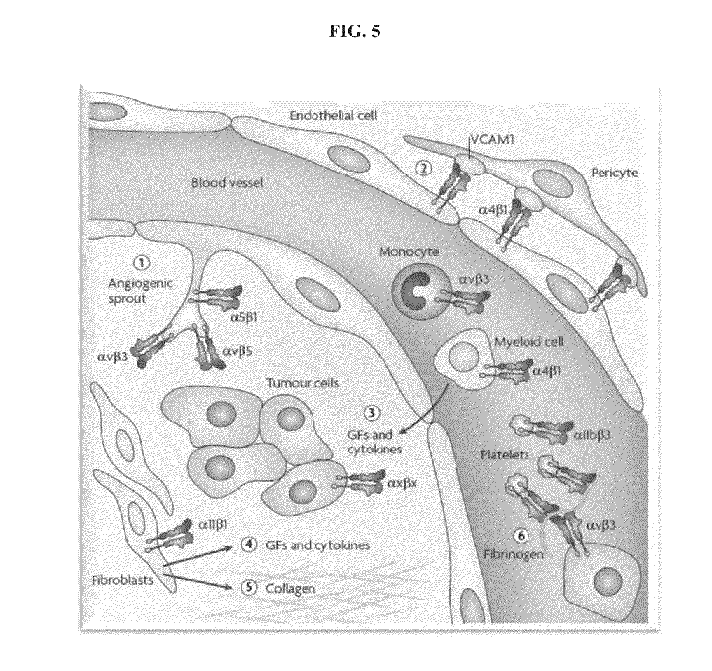

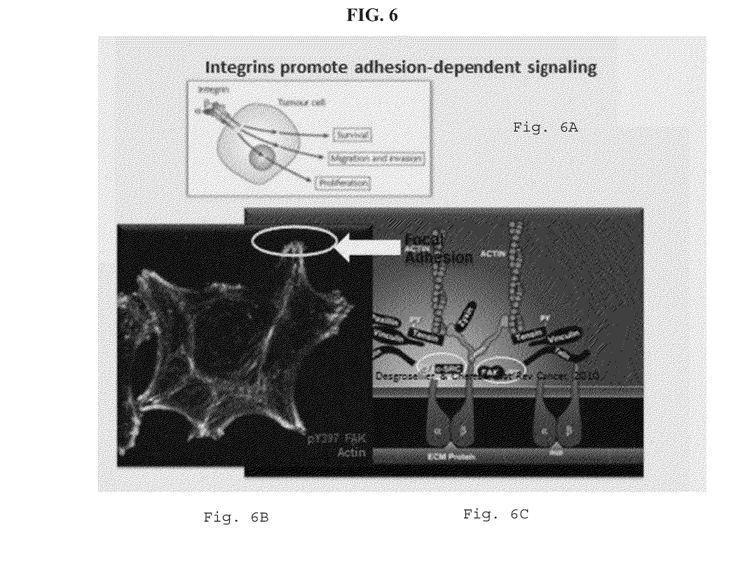

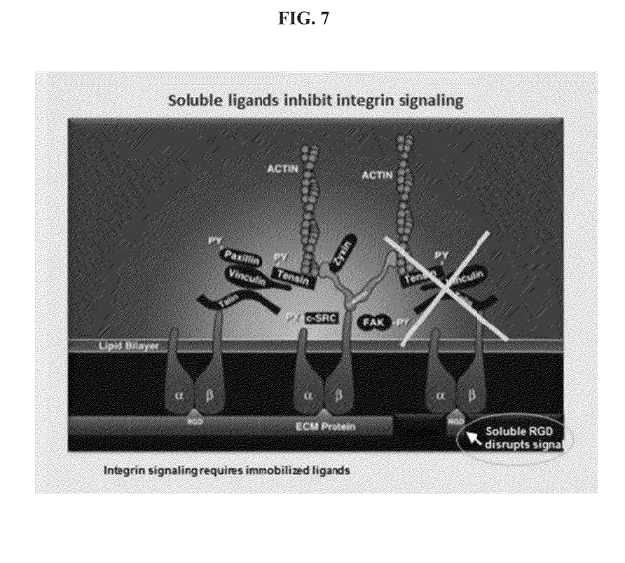

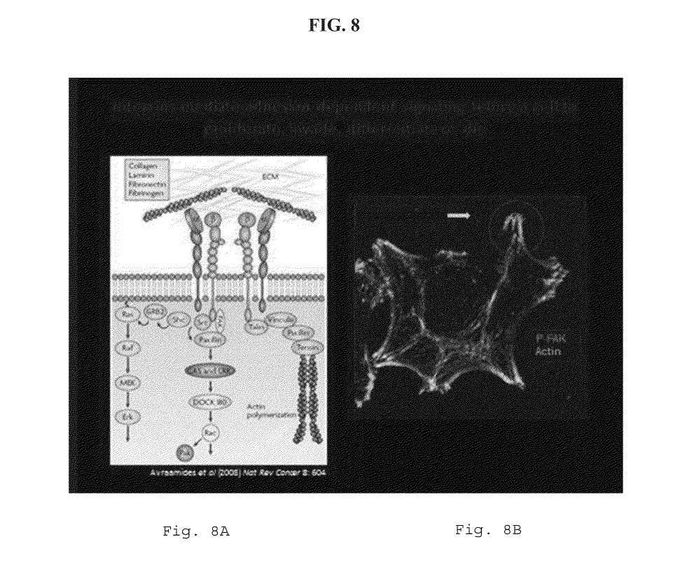

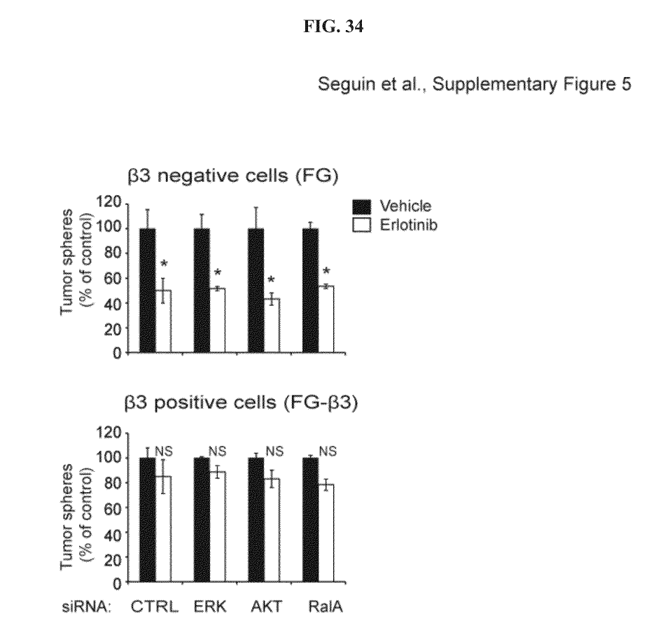

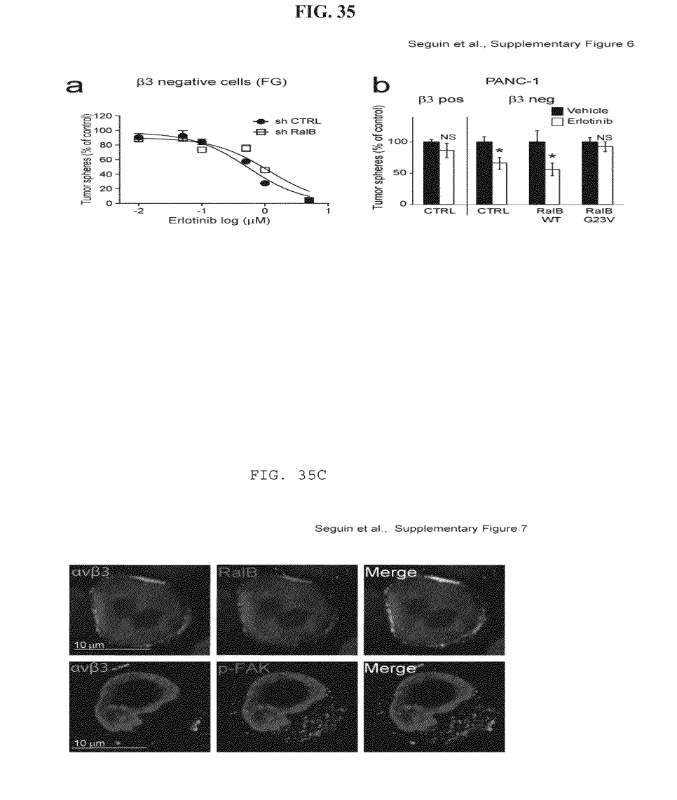

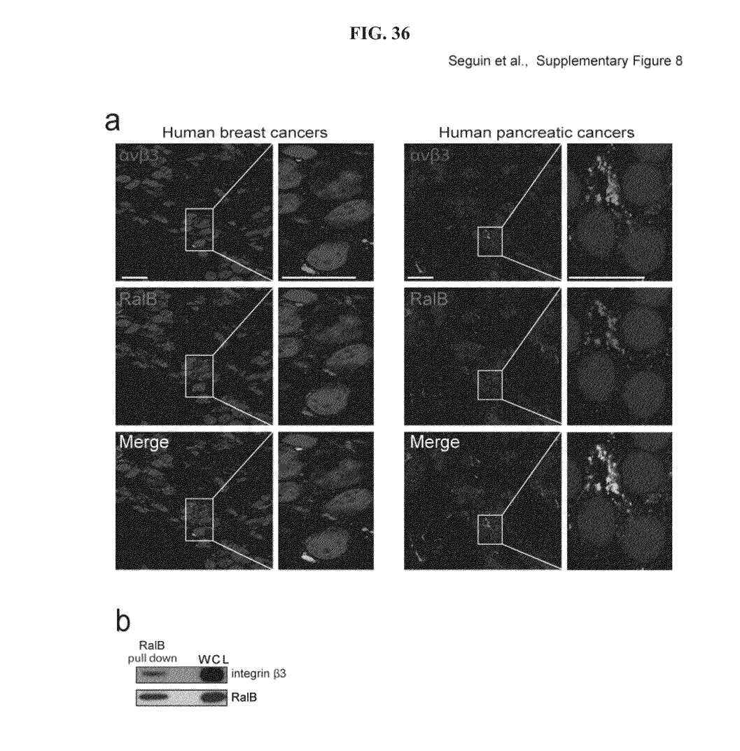

KRAS promotes multiple effector pathways including those regulated by RAF, phosphatidylinositol-3-OH kinases (PI3Ks) and RalGEFs leading to a variety of cellular functions²⁴. To investigate whether one or more KRAS effector pathway(s) may contribute to integrin β3/KRAS-mediated tumor cell resistance to EGFR inhibitors, we individually knocked-down or inhibited each downstream RAS effector in cells expressing or lacking integrin αvβ3. While suppression of AKT, ERK and RalA sensitized tumor cells to erlotinib, regardless of the αvβ3 expression status, see FIG. 34 (Supplementary FIG. 5), knockdown of RalB selectively sensitized αvβ3 expressing tumor cells to erlotinib, see FIG. 32a and FIG. 35a (Supplementary FIG. 6a). This was relevant to pancreatic tumor growth in vivo since, knockdown of RalB re-sensitized αvβ3-expressing pancreatic orthotopic tumors to erlotinib in mice, see FIG. 32b. In fact, expression of a constitutively active RalB (G23V) mutant in β3-negative cells was sufficient to confer resistance to EGFR inhibition, see FIG. 32c and FIG. 35b (Supplementary FIG. 6b). Furthermore, ectopic expression of αvβ3 enhanced RalB activity in tumor cells in a KRAS-dependent manner, see FIG. 32d). Accordingly, integrin αvβ3 and RalB were co-localized in tumor cells, see FIG. 35c (Supplementary FIG. 7) and in human breast and pancreatic cancer biopsies, see FIG. 36 (Supplementary FIG. 8) and a strong correlation was found between αvβ3 expression and Ral GTPase activity in patients biopsies suggesting the αvβ3/RalB signaling module is clinically relevant, see FIG. 32e. Together, these findings indicate that integrin αvβ3 promotes erlotinib resistance of cancer cells by complexing with KRAS and RalB resulting in RalB activation.

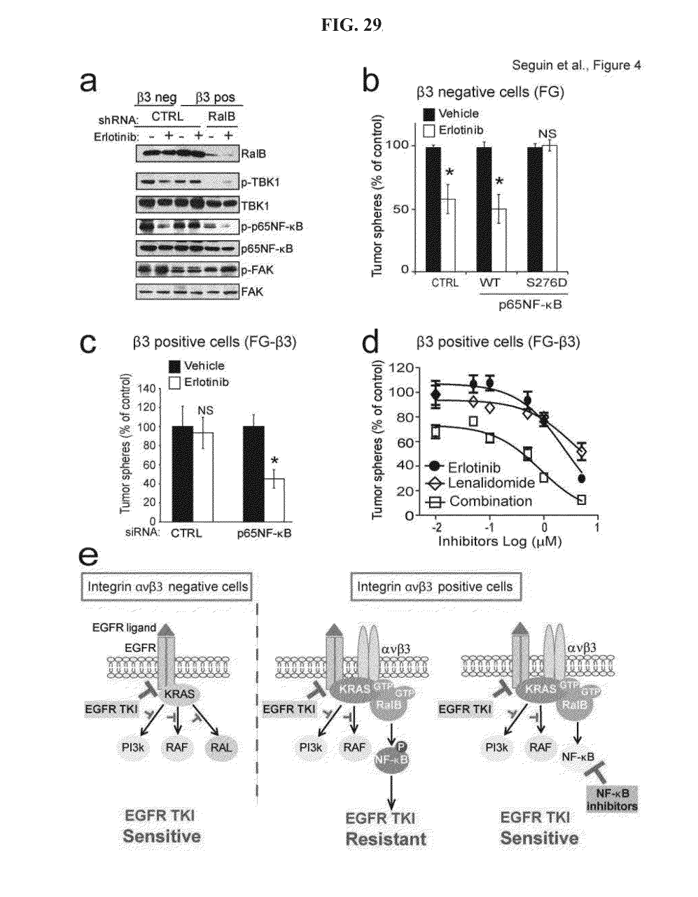

RalB, an effector of RAS has been shown to induce TBK1/NF-κB activation leading to enhanced tumor cell survival^{25,26}. In addition, it has been shown that NF-κB signaling is essential for KRAS-driven tumor growth and resistance to EGFR blockade²⁷⁻²⁹. This prompted us to ask whether αvβ3 could regulate NF-κB activity through RalB activation and thereby promote tumor cell resistance to EGFR targeted therapy. To test this, tumor cells expressing or lacking integrin αvβ3 and/or RalB were grown in the presence or absence of erlotinib and lysates of these cells were analyzed for activated downstream effectors of RalB. We found that erlotinib treatment of αvβ3 negative cells reduced levels of phosphorylated TBK1 and NF-κB, whereas in β3-positive cells these effectors remained activated unless RalB was depleted, see FIG. 29a. NF-κB activity was sufficient to account for EGFR inhibitor resistance since ectopically expressed a constitutively active NF-κB (S276D) in β3-negative FG pancreatic tumor cells³⁰ conferred resistance to EGFR inhibition, see FIG. 29b). Accordingly, genetic or pharmacological inhibition of NF-κB in β3-positive cells completely restored erlotinib sensitivity³¹, see FIGS. 29c and d). These findings demonstrate that RalB, the effector of the αvβ3/KRAS complex, promotes tumor cell resistance to EGFR targeted therapy via TBK1/NF-κB activation. Together, our studies describe a role for αvβ3 mediating resistance to EGFR inhibition via RalB activation and its downstream effector NF-κB, opening new avenues to target tumors that are resistant to EGFR targeted therapy, see FIG. 29e.

Recent studies have shown that, upon prolonged treatment with EGFR inhibitors, tumor cells develop alternative or compensatory pathways to sustain cell survival, leading to drug resistance^{1,32}. Here we show that integrin αvβ3 is specifically upregulated in histologically distinct tumors where it accounts for resistance to EGFR inhibition. At present, it is not clear whether exposure to EGFR inhibitors may promote increased αvβ3 expression or whether these drugs simply eliminate cells lacking αvβ3 allowing the expansion of αvβ3-expressing tumor cells. Given that integrin αvβ3 is a marker of mammary stem cells¹⁵, it is possible that acquired resistance to EGFR inhibitors selects for a tumor stem-like cell population^{3,33}. While integrins can promote adhesion dependent cell survival and induce tumor progression¹⁶, here, we show that integrin αvβ3, even in the unligated state, can drive tumor cell survival and resistance to EGFR blockade by interaction with KRAS. This action leads to the recruitment and activation of RalB and its downstream signaling effector NF-κB. In fact, NF-κB inhibition re-sensitizes αvβ3-bearing tumors to EGFR blockade. Taken together, our findings not only identify αvβ3 as a tumor cell marker of drug resistance but reveal that inhibitors of EGFR and NF-κB should provide synergistic activity against a broad range of cancers.

### FIGURE LEGENDS

FIG. 26.

Integrin αvβ3 expression promotes resistance to EGFR TKI.

(a) Flow cytometric quantification of cell surface markers after 3 weeks treatment with erlotinib (pancreatic and colon cancer cells) or lapatinib (breast cancer cells). (b) Flow cytometric analysis of αvβ3 expression in FG and Miapaca-2 cells following erlotinib. Error bars represent s.d. (n=3 independent experiments). (c) Top, immunofluorescence staining of integrin αvβ3 in tissue specimens obtained from orthotopic pancreatic tumors treated with vehicle (n=3) or erlotinib (n=4). Scale bar, 50 μm. Bottom, Integrin αvβ3 expression was quantified as ratio of integrin αvβ3 pixel area over nuclei pixel area using Metamorph (*P=0.049 using Mann-Whitney U test). (d) Right, intensity (scale 0 to 3) of β3 expression in mouse orthotopic lung tumors treated with vehicle (n=8) or erlotinib (n=7). Left, immunohistochemical staining of β3. Scale bar, 100 μm. (**P=0.0012 using Mann-Whitney U test) (e) IC₅₀ for cells treated with erlotinib or lapatinib. (f) Tumor sphere formation assay to establish a dose-response for erlotinib. Error bars represent s.d. (n=3 independent experiments). (g) Orthotopic FG tumors (>1000 mm³; n=10 per treatment group) were treated for 10 days with vehicle or erlotinib. Results are expressed as % tumor weight compared to vehicle control. *P<0.05. Immunoblot analysis for tumor lysates after 10 days of erlotinib confirms suppressed EGFR phosphorylation.

FIG. 27.

Integrin αvβ3 cooperates with KRAS to promote resistance to EGFR blockade.

(a-b) Tumor sphere formation assay of FG expressing (a) or lacking (b) integrin β3 depleted of KRAS (shKRAS) or not (shCTRL) and treated with a dose response of erlotinib. Error bars represent s.d. (n=3 independent experiments). (c) Confocal microscopy images of PANC-1 and FG-β3 cells grown in suspension. Cells are stained for integrin αvβ3 (green), KRAS (red), and DNA (TOPRO-3, blue). Scale bar, 10 □m. Data are representative of three independent experiments. (d) RAS activity assay performed in PANC-1 cells using GST-Raft-RBD immunoprecipitation as described in Methods. Immunoblot analysis of KRAS, NRAS, HRAS, RRAS, integrin β1 and integrin β3. Data are representative of three independent experiments. (e) Immunoblot analysis of Integrin αvβ3 immunoprecipitates from BxPC-3 β3-positive cells grown in suspension and untreated or treated with EGF 50 ng/ml for 5 minutes. RAS activity was determined using a GST-Raf1-RBD immunoprecipitation assay. Data are representative of three independent experiments.

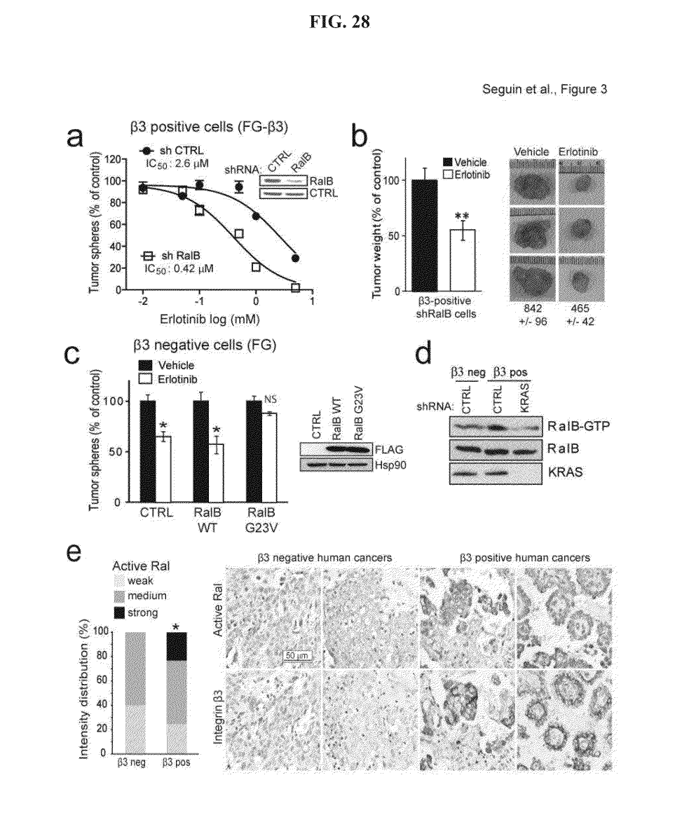

FIG. 28.

RalB is a key modulator of integrin αvβ3-mediated EGFR TKI resistance.

(a) Tumor spheres formation assay of FG-β3 treated with non-silencing (shCTRL) or RalB-specific shRNA and exposed to a dose response of erlotinib. Error bars represent s.d. (n=3 independent experiments). Immunoblot analysis showing RalB knockdown. (b) Effects of depletion of RalB on erlotinib sensitivity in β3-positive tumor in a pancreatic orthotopic tumor model. Established β3-positive tumors expressing non-silencing (shCTRL) or RalB-specific shRNA (>1000 mm³; n=13 per treatment group) were randomized and treated for 10 days with erlotinib. Results are expressed as % of tumor weight changes after erlotinib treatment compared to control. *P<0.05, **P<0.01. Tumor images, average weights+/−s.e are shown. (c) Tumor spheres formation assay of FG cells ectopically expressing vector control, WT RalB FLAG tagged constructs or a constitutively active RalB G23V FLAG tagged treated with erlotinib (0.5 μM). Error bars represent s.d. (n=3 independent experiments). *P<0.05, NS=not significant. Immunoblot analysis showing RalB WT and RalB G23 FLAG tagged constructs transfection efficiency. (d) RalB activity was determined in FG, FG-β3 expressing non-silencing or KRAS-specific shRNA, by using a GST-RalBP1-RBD immunoprecipitation assay as described in Methods. Data are representative of three independent experiments. (e) Right, overall active Ral immunohistochemical staining intensity between β3 negative (n=15) and β3 positive (n=70) human tumors. Active Ral staining was compared between each group by Fisher's exact test (*P<0.05, P=0.036, two-sided). Left, representative immunohistochemistry images of human tumor tissues stained with an integrin β3-specific antibody and an active Ral antibody. Scale bar, 50 μm.

FIG. 29.

Integrin αvβ3/RalB complex leads to NF-μB activation and resistance to EGFR TKI.

Immunoblot analysis of FG, FG-β3 and FG-β3 stably expressing non-silencing or RalB-specific ShRNA, grown in suspension and treated with erlotinib (0.5 μM). pTBK1 refers to phospho-S172 TBK1, p-p65 NF-κB refers to phospho-p65 NF-κB S276, pFAK refers to phospho-FAK Tyr 861. Data are representative of three independent experiments. (b) Tumor spheres formation assay of FG cells ectopically expressing vector control, WT NF-κB FLAG tagged or constitutively active S276D NF-κB FLAG tagged constructs treated with erlotinib (0.5 μM). Error bars represent s.d. (n=3 independent experiments). *P<0.05, **P<0.001, NS=not significant. Immunoblot analysis showing NF-κB WT and S276D NF-κB FLAG transfection efficiency. (c) Tumor spheres formation assay of FG-β3 treating with non-silencing (shCTRL) or NF-κB-specific shRNA and exposed to erlotinib (0.5 μM). Error bars represent s.d. (n=3 independent experiments). *P<0.05, NS=not significant. (d) Dose response in FG-β3 cells treated with erlotinib (10 nM to 5 μM), lenalidomide (10 nM to 5 μM) or a combination of erlotinib (10 nM to 5 μM) and lenalidomide (1 μM). Error bars represent s.d. (n=3 independent experiments). *P<0.05, NS=not significant. (e) Model depicting the integrin αvβ3-mediated EGFR TKI resistance and conquering EGFR TKI resistance pathway and its downstream RalB and NF-κB effectors.

### METHODS

Compounds and Cell Culture.

Human pancreatic (FG, PANC-1, Miapaca-2 (MP2), CFPAC-1, XPA-1, CAPAN-1, BxPc3), breast (MDAMB231, MDAMB468 (MDA468), BT20, SKBR3, BT474), colon (SW480) and lung (A549, H441) cancer cell lines were grown in ATCC recommended media supplemented with 10% fetal bovine serum, glutamine and non-essential amino acids. We obtained FG-β3, FG-D119A mutant and PANC-shβ3 cells as previously described¹⁷. Erlotinib, OSI-906, Gemcitabine and Lapatinib were purchased from Chemietek. Cisplatin was generated from Sigma-Aldrich. Lenalidomide was purchased from LC Laboratories. We established acquired EGFR TKI resistant cells by adding an increasing concentration of erlotinib (50 nM to 15 μM) or lapatinib (10 nM to 15 μM), daily in 3D culture in 0.8% methylcellulose.

Lentiviral Studies and Transfection.

Cells were transfected with vector control, WT, G23V RalB-FLAG, WT and S276D NF-κB-FLAG using a lentiviral system. For knock-down experiments, cells were transfected with KRAS, RalA, RalB, AKT1, ERK1/2, p65 NF-κB siRNA (Qiagen) using the lipofectamine reagent (Invitrogen) following manufacturer's protocol or transfected with shRNA (Open Biosystems) using a lentiviral system. Gene silencing was confirmed by immunoblots analysis.

Tumor Sphere Formation.

Tumor spheres formation assays were performed essentially as described previously¹⁷. Briefly, cells were seeded at 1000 to 2000 cells per well and grown for 12 days to 3 weeks. Cells were treated with vehicle (DMSO), erlotinib (10 nM to 5 μM), lapatinib (10 nM to 5 μM), gemcitabine (0.001 nM to 5 μM), OSI-906 (10 nM to 5 μM), lenalidomide (10 nM to 5 μM), or cisplatin (10 nM to 5 μM), diluted in DMSO. The media was replaced with fresh inhibitor every day for erlotinib, lapatinib, lenalidomide and 3 times a week for cisplatin and gemcitabine. Colonies were stained with crystal violet and scored with an Olympus SZH10 microscope. Survival curves were generated at least with five concentration points.

Flow Cytometry.

200,000 cells, after drug or vehicle treatment, were washed with PBS and incubated for 20 minutes with the Live/Dead reagent (Invitrogen) according to the manufacturer's instruction, then, cells were fixed with 4% paraformaldehyde for 15 min and blocked for 30 min with 2% BSA in PBS. Cells were stained with fluorescent-conjugated antibodies to CD61 (LM609), CD44 (eBioscience), CD24 (eBioscience), CD34 (eBioscience), CD133 (Santa Cruz), CD56 (eBioscience), CD29 (P4C10) and CD49f (eBioscience). All antibodies were used at 1:100 dilutions, 30 minutes at 4° C. After washing several times with PBS, cells were analyzed by FACS.

Immunohistochemical Analysis.

Immunostaining was performed according to the manufacturer's recommendations (Vector Labs) on 5 μM sections of paraffin-embedded tumors from the orthotopic xenograft pancreas and lung cancer mouse models¹⁴ or from a metastasis tissue array purchased from US Biomax (MET961). Antigen retrieval was performed in citrate buffer pH 6.0 at 95° C. for 20 min. Sections were treated with 0.3% H₂O₂ for 30 min, blocked in normal goat serum, PBS-T for 30 min followed by Avidin-D and then incubated overnight at 4° C. with primary antibodies against integrin β3 (Abcam) and active Ral (NewEast) diluted 1:100 and 1:200 in blocking solution. Tissue sections were washed and then incubated with biotinylated secondary antibody (1:500, Jackson ImmunoResearch) in blocking solution for 1 h. Sections were washed and incubated with Vectastain ABC (Vector Labs) for 30 min. Staining was developed using a Nickel-enhanced diamino-benzidine reaction (Vector Labs) and sections were counter-stained with hematoxylin. Sections stained with integrin β3 and active Ral were scored by a H-score according to the staining intensity (SI) on a scale 0 to 3 within the whole tissue section.

Immunoprecipitation and Immunoblot Analysis.

Cells were lysed in either RIPA lysis buffer (50 mM Tris pH 7.4, 100 mM NaCL, 2 mM EDTA, 10% DOC, 10% Triton, 0.1% SDS) or Triton lysis buffer (50 mM Tris pH 7.5, 150 mN NaCl, 1 mM EDTA, 5 mM MgCl2, 10% Glycerol, 1% Triton) supplemented with complete protease and phosphatase inhibitor mixtures (Roche) and centrifuged at 13,000 g for 10 min at 4° C. Protein concentration was determined by BCA assay. 500 μg to 1 mg of protein were immunoprecipitated with 3 μg of anti-integrin αvβ-3 (LM609) overnight at 4° C. following by capture with 25 μl of protein A/G (Pierce). Beads were washed five times, eluted in Laemmli buffer, resolved on NuPAGE 4-12% Bis-Tris Gel (Invitrogen) and immunoblotting was performed with anti-integrin β3 (Santa Cruz), anti-RalB (Cell Signaling Technology), anti KRAS (Santa Cruz). For immunoblot analysis, 25 μg of protein was boiled in Laemmli buffer and resolved on 8% to 15% gel. The following antibodies were used: KRAS (Santa Cruz), NRAS (Santa Cruz), RRAS (Santa Cruz), HRAS (Santa Cruz), phospho-S 172 NAK/TBK1 (Epitomics), TBK1 (Cell Signaling Technology), phospho-p65NF-κB S276 (Cell Signaling Technology), p65NF-κB (Cell Signaling Technology), RalB(Cell Signaling Technology), phospho-EGFR (Cell Signaling Technology), EGFR (Cell Signaling Technology), FLAG (Sigma), phospho-FAK Tyr 861 (Cell Signaling Technology), FAK (Santa Cruz), Galectin 3 (BioLegend) and Hsp90 (Santa Cruz).

Affinity Pull-Down Assays for Ras and Ral.

RAS and Ral activation assays were performed in accordance with the manufacturer's (Upstate) instruction. Briefly, cells were cultured in suspension for 3 h, lysed and protein concentration was determined. 10 μg of Ral Assay Reagent (Ral BP1, agarose) or RAS assay reagent (Raf-1 RBD, agarose) was added to 500 mg to 1 mg of total cell protein in MLB buffer (Millipore). After 30 min of rocking at 4° C., the activated (GTP) forms of RAS/Ral bound to the agarose beads were collected by centrifugation, washed, boiled in Laemmli buffer, and loaded on a 15% SDS-PAGE gel.

Immunofluorescence Microscopy.

Frozen sections from tumors from the orthotopic xenograft pancreas cancer mouse model or from patients diagnosed with pancreas or breast cancers (as approved by the institutional Review Board at University of California, San Diego) or tumor cell lines were fixed in cold acetone or 4% paraformaldehyde for 15 min, permeabilized in PBS containing 0.1% Triton for 2 min and blocked for 1 h at room temperature with 2% BSA in PBS. Cells were stained with antibodies to integrin αvβ3 (LM609), RalB (Cell Signaling Technology), Galectin 3 (BioLegend), pFAK (Cell Signaling Technology), NRAS (Santa Cruz), RRAS (Santa Cruz), HRAS (Santa Cruz) and KRAS (Abgent). All primary antibodies were used at 1:100 dilutions, overnight at 4° C. Where mouse antibodies were used on mouse tissues, we used the MOM kit (Vector Laboratory). After washing several times with PBS, cells were stained for two hours at 4° C. with secondary antibodies specific for mouse or rabbit (Invitrogen), as appropriate, diluted 1:200 and co-incubated with the DNA dye TOPRO-3 (1:500) (Invitrogen). Samples were mounted in VECTASHIELD hard-set media (Vector Laboratories) and imaged on a Nikon Eclipse C1 confocal microscope with 1.4 NA 60× oil-immersion lens, using minimum pinhole (30 □m). Images were captured using 3.50 imaging software. Colocalization between Integrin αvβ3 and KRAS was studied using the Zenon Antibody Labeling Kits (Invitrogen).

Orthotopic Pancreas Cancer Xenograft Model.

All mouse experiments were carried out in accordance with approved protocols from the UCSD animal subjects committee and with the guidelines set forth in the NIH Guide for the Care and Use of Laboratory Animals. Tumors were generated by injection of FG human pancreatic carcinoma cells (10⁶ tumor cells in 30 □L of sterile PBS) into the tail of the pancreas of 6-8 week old male immune compromised nu/nu mice. Tumors were established for 2-3 weeks (tumor sizes were monitored by ultrasound) before beginning dosing. Mice were dosed by oral gavage with vehicle (6% Captisol) or 100 mg/kg/day erlotinib for 10 to 30 days prior to harvest.

Orthotopic Lung Cancer Xenograft Model.

Tumors were generated by injection of H441 human lung adenocarcinoma cells (10⁶ tumor cells per mouse in 50 μL of HBSS containing 50 mg growth factor-reduced Matrigel (BD Bioscience) into the left thorax at the lateral dorsal axillary line and into the left lung, as previously described¹⁴ of 8 week old male immune-compromised nu/nu mice. 3 weeks after tumor cell injection, the mice were treated with vehicle or erlotinib (100 mg/kg/day) by oral gavage until moribund (approximately 50 and 58 days, respectively).

Statistical Analyses.

All statistical analyses were performed using Prism software (GraphPad). Two-tailed Mann Whitney U tests, Fisher's exact tests, or t-tests were used to calculate statistical significance. A P value <0.05 was considered to be significant.

### REFERENCES

1. Wheeler, D. L., Dunn, E. F. & Harari, P. M. Understanding resistance to EGFR inhibitors-impact on future treatment strategies. Nat Rev Clin Oncol 7, 493-507 (2010).
2. Dorans, K. Outpacing cancer. Nature Medicine 15, 718-722 (2009).
3. Dean, M., Fojo, T. & Bates, S. Tumour stem cells and drug resistance. Nature Reviews 5, 275-284 (2005).
4. Engelman, J. A. & Janne, P. A. Mechanisms of acquired resistance to epidermal growth factor receptor tyrosine kinase inhibitors in non-small cell lung cancer. Clin Cancer Res 14, 2895-2899 (2008).
5. Montagut, C., et al. Identification of a mutation in the extracellular domain of the Epidermal Growth Factor Receptor conferring cetuximab resistance in colorectal cancer. Nature Medicine 18, 221-223 (2012).
6. Sharma, S. V., Bell, D. W., Settleman, J. & Haber, D. A. Epidermal growth factor receptor mutations in lung cancer. Nature Reviews 7, 169-181 (2007).
7. Zoppoli, G., et al. Ras-induced resistance to lapatinib is overcome by MEK inhibition. Current Cancer Drug Targets 10, 168-175 (2010).
8. Gupta, S., et al. Binding of ras to phosphoinositide 3-kinase p110alpha is required for ras-driven tumorigenesis in mice. Cell 129, 957-968 (2007).
9. Lim, K. H., et al. Activation of RalA is critical for Ras-induced tumorigenesis of human cells. Cancer Cell 7, 533-545 (2005).
10. Sharma, S. V., et al. A chromatin-mediated reversible drug-tolerant state in cancer cell subpopulations. Cell 141, 69-80 (2010).
11. Liu, C., et al. The microRNA miR-34a inhibits prostate cancer stem cells and metastasis by directly repressing CD44. Nature Medicine 17, 211-215 (2011).
12. Vaillant, F., et al. The mammary progenitor marker CD61/beta3 integrin identifies cancer stem cells in mouse models of mammary tumorigenesis. Cancer Research 68, 7711-7717 (2008).
13. Adhikari, A. S., Agarwal, N. & Iwakuma, T. Metastatic potential of tumor-initiating cells in solid tumors. Front Biosci 16, 1927-1938 (2011).
14. Cascone, T., et al. Upregulated stromal EGFR and vascular remodeling in mouse xenograft models of angiogenesis inhibitor-resistant human lung adenocarcinoma. J Clin Invest 121, 1313-1328 (2011).
15. Asselin-Labat, M. L., et al. Gata-3 is an essential regulator of mammary-gland morphogenesis and luminal-cell differentiation. Nature Cell Biology 9, 201-209 (2007).
16. Desgrosellier, J. S. & Cheresh, D. A. Integrins in cancer: biological implications and therapeutic opportunities. Nature Reviews 10, 9-22 (2010).
17. Desgrosellier, J. S., et al. An integrin alpha(v)beta(3)-c-Src oncogenic unit promotes anchorage-independence and tumor progression. Nature Medicine 15, 1163-1169 (2009).
18. Borst, P., Jonkers, J. & Rottenberg, S. What makes tumors multidrug resistant?Cell Cycle 6, 2782-2787 (2007).
19. Schmitt, C. A., et al. A senescence program controlled by p53 and p16INK4a contributes to the outcome of cancer therapy. Cell 109, 335-346 (2002).
20. Baselga, J. & Rosen, N. Determinants of RASistance to anti-epidermal growth factor receptor agents. J Clin Oncol 26, 1582-1584 (2008).
21. Moore, M. J., et al. Erlotinib plus gemcitabine compared with gemcitabine alone in patients with advanced pancreatic cancer: a phase III trial of the National Cancer Institute of Canada Clinical Trials Group. J Clin Oncol 25, 1960-1966 (2007).
22. Levy, R., Grafi-Cohen, M., Kraiem, Z. & Kloog, Y. Galectin-3 promotes chronic activation of K-Ras and differentiation block in malignant thyroid carcinomas. Molecular Cancer Therapeutics 9, 2208-2219 (2010).
23. Markowska, A. I., Liu, F. T. & Panjwani, N. Galectin-3 is an important mediator of VEGF- and bFGF-mediated angiogenic response. The Journal of Experimental Medicine 207, 1981-1993 (2010).
24. Buday, L. & Downward, J. Many faces of Ras activation. Biochim Biophys Acta 1786, 178-187 (2008).
25. Barbie, D. A., et al. Systematic RNA interference reveals that oncogenic KRAS-driven cancers require TBK1. Nature 462, 108-112 (2009).
26. Chien, Y., et al. RalB GTPase-mediated activation of the IkappaB family kinase TBK1 couples innate immune signaling to tumor cell survival. Cell 127, 157-170 (2006).
27. Ling, J., et al. Kras(G12D)-Induced 1KK2/beta/NF-kappaB Activation by IL-1 alpha and p62 Feedforward Loops Is Required for Development of Pancreatic Ductal Adenocarcinoma. Cancer Cell 21, 105-120 (2012).
28. Bivona, T. G., et al. FAS and NF-kappaB signalling modulate dependence of lung cancers on mutant EGFR. Nature 471, 523-526 (2011).
29. Min, J., et al. An oncogene-tumor suppressor cascade drives metastatic prostate cancer by coordinately activating Ras and nuclear factor-kappaB. Nature Medicine 16, 286-294 (2010).
30. Dong, J., Jimi, E., Zeiss, C., Hayden, M. S. & Ghosh, S. Constitutively active NF-kappaB triggers systemic TNFalpha-dependent inflammation and localized TNFalpha-independent inflammatory disease. Genes & Development 24, 1709-1717 (2011).
31. Braun, T., et al. Targeting NF-kappaB in hematologic malignancies. Cell Death Differ 13, 748-758 (2006).
32. Workman, P. & Clarke, P. A. Resisting targeted therapy: fifty ways to leave your EGFR. Cancer Cell 19, 437-440 (2011).
33. Lewis, M. T. & Wicha, M. S. Tumor-initiating cells and treatment resistance: how goes the war? Journal of Mammary Gland Biology and Neoplasia 14, 1-2 (2009).

A number of embodiments of the invention have been described. Nevertheless, it will be understood that various modifications may be made without departing from the spirit and scope of the invention. Accordingly, other embodiments are within the scope of the following claims.

1-4. (canceled)

5. A method for determining the responsiveness of an individual to a treatment comprising blocking activation of an alphaᵥ-beta₃ (or αᵥ-β₃) integrin polypeptide, or blocking the interaction of a ligand with an alphaᵥ-beta₃ (or αᵥ-β₃) integrin polypeptide, or blocking the phosphorylation of a C-RAF polypeptide, comprising:
(a) identifying or determining the phosphorylation state of a C-RAF serine residue 338 (ser-338) on a C-RAF polypeptide, or identifying or determining that a C-RAF serine residue 338 (ser-338) is phosphorylated, or identifying or determining the extent to which cellular C-RAFs are ser-338 phosphorylated, determines the responsiveness of an individual to the treatment;
(b) identifying or determining the phosphorylation state of a C-RAF serine residue 338 (ser-338) on a C-RAF polypeptide, or identifying or determining that a C-RAF serine residue 338 (ser-338) is phosphorylated, or identifying or determining the extent to which cellular C-RAFs are ser-338 phosphorylated, at two different time points, and if the amount of phosphorylation of ser-338 decreases in the second time point relative to the first time point, the individual is determined to be responsive to the treatment; or
(c) the method of (a) or (b), wherein the method is prognostic in that individuals or patients having decreased levels or amounts of phosphorylated ser-338 are determined or predicted to survive longer.

6-7. (canceled)

8. A method for:
arresting a proliferating tumor cell at prometaphase by reducing or inhibiting the activity of a human P21 protein (Cdc42/Rac)-Activated Kinase (PAK or c-PAK);
reducing or inhibiting serine 338 (Ser 338) phosphorylation of a c-RAF;
reducing or inhibiting a c-RAF-dependent dysfunctional cell, cancer cell or tumor growth;
promoting a tumor regression in vivo in a c-RAF-dependent human tumor or cancer cell;
inducing double-stranded DNA breakage in a cell; or,
sensitizing a tumor cell to a radiation (radiosensitizing a cell) or a chemotherapy; comprising
(1) (a) providing a composition comprising or consisting of:
(i) an inhibitor of a PAK (or c-PAK) protein activity, or
(ii) the PAK-inhibiting composition of (i), wherein the PAK inhibitor comprises a small molecule, an antibody, a dominant negative PAK inhibitor, a siRNA, an miRNA, or an antisense oligonucleotide; and
(b) administering a sufficient amount of the composition to a cell or a subject to reduce or inhibit the activity of the PAK kinase, or human PAK kinase,
wherein optionally administering the PAK inhibitor comprises arresting a proliferating tumor cell at prometaphase,
wherein optionally administering the PAK inhibitor comprises reducing or inhibiting a serine 338 (Ser 338) phosphorylation of a c-RAF,
wherein optionally administering the PAK inhibitor reduces or inhibits a c-RAF-dependent dysfunctional cell, cancer cell or tumor growth,
wherein optionally administering the PAK inhibitor promotes a tumor regression in vivo in a c-RAF-dependent human tumor or cancer cell,
wherein optionally administering the PAK inhibitor induces double-stranded DNA breakage in a cell, or sensitizes a tumor cell to a radiation or a chemotherapy; or
(2) the method of (1), wherein the composition comprises a pharmaceutical composition formulated for administration in vivo;
(3) the method of (1) or (2), wherein the composition is formulated for administration intravenously (IV), parenterally, nasally, topically, orally, or by liposome or vessel-targeted nanoparticle delivery;
(4) the method of any of (1) to (3), wherein the composition comprises a pharmaceutical composition administered in vivo;
(5) the method of any of (1) to (3), wherein the administration comprises contacting a cell or tumor in vitro or ex vivo;
(6) the method of any of (1) to (5), wherein the dominant-negative peptide PAK inhibitor comprises a peptidomimetic;
(7) the method of any of (1) to (5), wherein the PAK inhibitor comprises or consists of a peptide having a sequence HTIHVGFDAV TGEFTGMPEQ WARLLQTSNI TKSEQKKNPQ AVLDVLEFYN SKKTSNSQKY MSFTDKS (SEQ ID NO:1);
(8) the method of any of (1) to (5), wherein the antibody PAK inhibitor comprises or is a monoclonal antibody, a humanized antibody or a human antibody, or an antigen-binding (PAK-binding) fragment thereof; or
(9) the method of any of (1) to (8), wherein the method reduces, treats or ameliorates the level of disease in a retinal age-related macular degeneration, a diabetic retinopathy, a cancer or carcinoma, a glioblastoma, a neuroma, a neuroblastoma, a colon carcinoma, a hemangioma, an infection and/or a condition with at least one inflammatory component, and/or any infectious or inflammatory disease, such as a rheumatoid arthritis, a psoriasis, a fibrosis, leprosy, multiple sclerosis, inflammatory bowel disease, or ulcerative colitis or Crohn's disease.

9-23. (canceled)

24. A combination, or a therapeutic combination, for overcoming or diminishing or preventing Growth Factor Inhibitor (GFI) resistance in a cell, or, a method for increasing the growth-inhibiting effectiveness of a Growth Factor inhibitor on a cell, or, a method for re-sensitizing a cell to a Growth Factor Inhibitor (GFI), wherein the combination comprises or consists of:
(1) at least one compound comprising or consisting of:
(i) an inhibitor or depleter of integrin αᵥβ₃ (anb3), or an inhibitor of integrin αᵥβ₃ (anb3) protein activity, or an inhibitor of the formation or activity of an integrin anb3/RalB signaling complex, or an inhibitor of the formation or signaling activity of an integrin αᵥβ₃ (anb3)/RalB/NFkB signaling axis,
wherein the inhibitor of integrin αᵥβ₃ protein activity is an allosteric inhibitor of integrin αᵥβ₃ protein activity;
(ii) an inhibitor or depleter of RalB protein or an inhibitor of RalB protein activation,
wherein the inhibitor of RalB protein activity is an allosteric inhibitor of RalB protein activity;
(iii) an inhibitor or depleter of Src or TBK1 protein or an inhibitor of Src or TBK1 protein activation,
wherein the inhibitor of Src or TBK1 protein activity is an allosteric inhibitor of Src or TBK1 protein activity;
(iv) an inhibitor or depleter of NFKB or IRF3 protein or an inhibitor of RalB protein activation,
wherein the inhibitor of NFKB or IRF3 protein activity is an allosteric inhibitor of NFKB or IRF3 protein activity; or
(v) any combination of (i) to (iv); and
(2) at least one Growth Factor Inhibitor;
wherein optionally the cell is a tumor cell, a cancer cell, a cancer stem cell, or a dysfunctional cell.


SEQUENCE LISTING (1 sequence)

SEQ ID NO: 1 - protein, 67 aa, artificial sequence
  synthetic peptide
HTIHVGFDAVTGEFTGMPEQWARLLQTSNITKSEQKKNPQAVLDVLEFYNSKKTSNSQKYMSFTDKS